(19) United States
(12) Patent Application Publication
(10) Pub. No.: US 2010/0120701 A1
(43) Pub. Date: May 13, 2010

(54) Compositions and Methods For Engineering Probiotic Yeast

(75) Inventors: John M. McCoy, Reading, MA (US); Phillips W. Robbins, Cambridge, MA (US)

Correspondence Address: MINTZ, LEVIN, COHN, FERRIS, GLOVSKY AND POPEO, P.C, ONE FINANCIAL CENTER, BOSTON, MA, 02111, US

(73) Assignee: Glycosyn, Inc., Medford, MA (US)

(21) Appl. No.: 12/567,135

(22) Filed: Sep. 25, 2009

Related U.S. Application Data
(60) Provisional application No. 61/194,161, filed on Sep. 25, 2008.

(51) Int. Cl. A61K 31/70; C12N 1/19; C12N 15/00; A61P 31/12

(52) U.S. Cl. 514/23; 435/254.2; 435/440

(57) ABSTRACT

The invention provides compositions and methods for engineering probiotic yeast to display α(1,2) fucosylated glycans on their cell surface, and the use thereof in the prevention or treatment of infection.

### FIELD OF THE INVENTION

The invention provides compositions and methods for engineering probiotic yeast that prevent infections through inhibition of pathogen adherence to epithelia.

### BACKGROUND OF THE INVENTION

Worldwide, infectious diarrhea is responsible for approximately 20% of all mortality in children under the age of 5, and for an estimated 2.5 millions deaths annually. In the United States, infectious diarrhea has an annual incidence of over 200 million cases and is responsible for approximately 900,000 hospitalizations and 5,000 deaths per year. Included in these numbers are 200,000 children under five years of age hospitalized in the U.S. each year with active diarrheal disease, accounting for nearly 880,000 inpatient days, over 500 deaths, and almost one billion dollars of inpatient cost.

### SUMMARY OF THE INVENTION

The invention provides compositions comprising a yeast cell, engineered to produce α(1,2) fucosylated glycans on its cell surface, in a probiotic formulation that promotes intestinal health by decreasing the adherence and or colonization of the gastrointestinal tract by pathogenic organisms. Any edible yeast cell is suitable host for expression of the glycans. Preferably, the genus of the yeast cell is Kluyveromyces. Kluyveromyces lactis is tolerant to conditions found in the human gut and may be particularly well suited to probiotic applications, although yeast of other genera could be used. For example, Saccharomyces yeast cells are used to display α(1,2) fucosylated glycans on the cell surface of a yeast cell. In one example, the formulation also contains probiotic bacteria such as Lactobacilli; alternatively, the formulation does not comprise bacterial probiotic strains.

The engineered yeast cell harbors a exogenous (recombinant) nucleic acid molecule encoding GDP-mannose-4,6-dehydratase (GMD) and an exogenous (recombinant) nucleic acid molecule encoding GDP-L-fucose synthetase (GFS). In one aspect, the GMD is selected from the group consisting of H. pylori GMD, E. coli GMD, and Homo sapiens GMD, and the GFS is selected from the group H. pylori GFS, E. coli GFS, and Homo sapiens GFS. Optionally, the composition further comprises a recombinant nucleic acid construct encoding GDP-mannose pyrophosphorylase (e.g. K. lactis PSA1) and directing increased expression of this enzyme. In addition the composition may optionally comprise recombinant nucleic acid constructs encoding phosphomannose isomerase (e.g. K. lactis locus KLLA0D13728g, PMI) and/or phosphomannose mutase (e.g. K. lactis SEC53, PMM), leading to increased expression of these enzymes. The composition optionally comprises a nucleic acid encoding phosphomannose isomerase and/or a nucleic acid molecule encoding phosphomannose mutase.

The invention also provides for a yeast cell that further harbors a nucleic acid molecule encoding a nucleotide sugar transporter (NST) (to ensure that the appropriate precursor molecules synthesized in the cell cytoplasm are made available in the golgi for incorporation into cell-surface glycans). In one aspect, the NST is selected from the group consisting of human FUCT1 (GDP-fucose transporter), mouse SLC35a2 (UDP-galactose transporter), human UGT1 (UDP-galactose transporter), Schizosaccharomyces pombe GMS 1 (UDP-galactose transporter), Saccharomyces cerevisiae HUT1 (UDP-galactose transporter). Optionally, the yeast cell over-expresses an endogenous NST.

In another aspect, the yeast cell lacks terminal N-acetylglucosaminyltransferase activity or UDP-N-acetylglucosamine transporter activity. Preferably, the yeast cell is a Kluyveromyces lactis yeast cell carrying mutations in the GNT1 (MNN2-1) (N-acetylglucosaminyltransferase) or MNN2 (MNN2-2) (UDP-N-acetylglucosamine transporter) genes.

The invention also provides a yeast cell that further harbors a nucleic acid molecule encoding an α(1,2) galactosyltransferase. Optionally, the nucleic acid molecule encoding an α(1,2) galactosyltransferase is Schizosaccharomyces pombe GMA12.

In another aspect, the yeast cell further harbors a nucleic acid molecule encoding a β(1,3) galactosyltransferase. Optionally, the nucleic acid molecule encoding a β(1,3) galactosyltransferase is Schizosaccharomyces pombe PVG3.

The invention also provides for a yeast cell that further harbors a recombinant nucleic acid molecule encoding a fucosyltransferase. Preferably, the fucosyltransferase is a human α(1,2) fucosyltransferase (FUT1 or FUT2).

Preferably, the yeast cell displays α(1,2) fucosylated glycans on its cell surface. More preferably, the yeast cell is a Kluyveromyces lactis yeast cell.

Exemplary protein (amino acid) sequences and GENBANK™ Accession Numbers, the listings of which also contain nucleic acid sequences encoding the reference protein are provided below.

H. pylori GMD:
>YP_003056882 GDP-mannose 4,6-dehydratase (GDP-D-
mannose dehydratase) [Heilcobacter pylori B38].

(SEQ ID NO: 1)

MKEKIALITGVTGQDGSYLAEYLLNLGYEVHGLKRRSSSINTSRIDHLYE
DLHSDHKRRFFLHYGDMTDSSNLIHLIATTKPTEIYNLAAQSHVKVSFET
PEYTANADGIGTLRILEAMRILGLEKKTRFYQASTSELYGEVLETPQNEN
TPFNPRSPYAVAKMYAFYITKNYREAYNLFAVNGILFNHESRVRGETFVT
RKITRAASTIAYNLTDCLYLGNLDAKRDWGHAKDYVKMMHLMLQAPTPQD
YVIATGKTTSVRDFVKMSFEFIGINLEFQNTGIKEIGLIKSVDEKRANAL
QLNLSHLKTGQIVVRIDEHYFRPTEVDLLLGDPTKAEKELGWVREYDLKE
LVKDMLEYDLKECQKNLYLQDGGYILRNFYE
E. coli GMD:
>AAL67553 GDP-mannose dehydratase Gmd [Escherichia
coli].

(SEQ ID NO: 2)

MTKVALITGVTGQDGSYLAEFLLEKGYEVHGIKRRASSFNTERVDHIYQD
PHSAKPNFHLHYGDLTDTSNITRILQEVKPDEVYNLGAMSHVAVSFESPE
YTADVDAIGTLRLLEAIRILGLEKKTRFYQASTSELYGLVQEIPQKETTP
FYPRSPYAVAKLYAYWITVNYRESYGIYACNGILFNHESPRRGETFVTRK
ITRAIANIAQGLESCLYLGNMDSLRDWGHAKDYVRMQWMMLQQEQPEDFV
IATGVQYSVREFVEMTAEQLGIKLSFEGKGVDEKGIVVSVTGDKAPGVKT
GDVIVAVDPRYFRPAEVETLLGDPAKAHEKLGWKPEITLREMISEMVAND
LQTAKKHVLLKSHGFNANLAQE
Homo sapiens GMD:
>EAW55075 GDP-mannose 4,6-dehydratase [Homo
sapiens].

(SEQ ID NO: 3)

MAHAPARCPSARGSGDGEMGKPRNVALITGITGQDGSYLAEFLLEKGYEV
HGIVRRSSSFNTGRIEHLYKNPQAHIEGNMKLHYGDLTDSTCLVKIINEV
KPTEIYNLGAQSHVKISFDLAEYTADVDGVGTLRLLDAVKTCGLINSVKF
YQASTSELYGKVQEIPQKETTPFYPRSPYGAAKLYAYWIVVNFREAYNLF
AVNGILFNHESPRRGANFVTRKISRSVAKIYLGQLECFSLGNLDAKRDWG
HAKDYVEAMWLMLQNDEPEDFVIATGEVHSVREFVEKSFLHIGKTIVWEG
KNENEVGRCKETGKVHVTVDLKYYRPTEVDFLQGDCTKAKQKLNWKPRVA
FDELVREMVHADVELMRTNPNA
H. pylori GFS:
>AAC64910 O-antigen biosynthesis protein
[Heilcobacter pylori].

(SEQ ID NO: 4)

MNEIILITGAYGMVGQNTALYFKKNKPDVTLLTPKKSELCLLDKDNVQAY
LKEYKPTGIIHCAGRVGGIVANMNDLSTYNVENLLMGLYLFSSALDLGVK
KAINLASSCAYPKYAPNPLKESDLLNGSLEPTNEGYALAKLSVMKYCEYV
STEKGGFYKTLVPCNLYGEFDKFEEKIAHMIPGLIARMHTAKLKNEKNFA
MWGDGTARREYLNAKDLARFIALAYENIASMPSVMNVGSGVDYSIEEYYE
MVAQVLDYKGVFVKDLSKPVGMQQKLMDISKQKALKWELEIPLEQGIKEA
YEYYLKLLEV
E. coli GFS:
>AAC77843 GDP-L-fucose synthetase [Escherichia
coli].

(SEQ ID NO: 5)

MSKQRVFIAGHRGMVGSAIRRQLEQRGDVELVLRTRDELNLLDSRAVHDF
FASERIDQVYLAAAKVGGIVANNTYPADFIYQNMMIESNIIHAAHQNDVN
KLLFLGSSCIYPKLAKQPMAESELLQGTLEPTNEPYAIAKIAGIKLCESY
NRQYGRDYRSVMPTNLYGPHDNFHPSNSHVIPALLRRFHEATAQNAPDVV
VWGSGTPMREFLHVDDMAAASIHVMELAHEVWLENTQPMLSHINVGTGVD
CTIRDVAQTIAKVVGYKGRVVFDASKPDGTPRKLLDVTRLHQLGWYHEIS
LEAGLASTYQWFLENQDRFRG
Homo sapiens GFS:
>Q13630 GDP-L-fucose synthetase [Home sapiens]

(SEQ ID NO: 6)

MGEPQGSMRILVTGGSGLVGKAIQKVVADGAGLPGEDWVFVSSKDADLTD
TAQTRALFEKVQPTHVIHLAAMVGGLFRNIKYNLDFWRKNVHMNDNVLHS
AFEVGARKVVSCLSTCIFPDKTTYPIDETMIHNGPPHNSNFGYSYAKRMI
DVQNRAYFQQYGCTFTAVIPTNVFGPHDNFNIEDGHVLPGLIHKVHLAKS
SGSALTVWGTGNPRRQFIYSLDLAQLFIWVLREYNEVEPIILSVGEEDEV
SIKEAAEAVVEAMDFHGEVTFDTTKSDGQFKKTASNSKLRTYLPDFRFTP
FKQAVKETCAWFTDNYEQARK
K. lactis PSA1:
>Q70SJ2 GDP-mannose pyrophosphorylase
[Kluyveromyces lactis].

(SEQ ID NO: 7)

MKGLILVGGYGTRLRPLTLTVPKPLVEFGNRPMILHQIEALAAAGVTDIV
LAVNYRPEVMVETLKKYEDEFGVSITFSVETEPLGTAGPLKLAESVLKKD
NSPFFVLNSDVICDYPFKELADFHQAHGGKGTIVATKVDEPSKYGVIVHD
IATPNLIDRFVEKPVEFVGNRINAGLYILNPEVIDLIDLKPTSIEKETFP
ILVEQKSLYSFDLEGYWMDVGQPKDFLSGTVLYLNSLSKRDPAKLAKGEN
IVGNVLVDPTAKISPTAKVGPDVVIGPNVVIGDGVRITRSVALSNSHIKD
HALVKSTIIGWNSTVGKWARLEGVTVLGDDVEVKDEIYINGGKVLPHKSI
SVNVPKEAIIM
K. lactis locus KLLA0D13728g, PMI:
>CAH00771 KLLA0D13728p phosphomannose isomerase
[Kluyveromyces lactis].

(SEQ ID NO: 8)

MPQLFRLDAGFQQYDWGKIGSSSAVAQFAAHSDPSVKIDEQKPYAELWMG
THHKVPSYNHDTKESLRDLIEADPVGMLGQGNVDKFGSMKELPFLFKVLS
IKKVLSIQAHPDKALAKVLHFNDPANYPDDNHKPEMALAVTDFEGFCGFK
PLEEIADELQRIPELRNIVGDEVSETFINNINPEAVKDSADDAKNKKLLQ
QVFSKVMNASDKVVVENARALIKRAHESPADFNKDTLPQLLIDLNEQFPD
DVGLFCGGLLLNHCNLKAGEAIFLRAKDPHAYISGDIIECMAASDNVVRA
GFTPKFKDVKNLVEMLTYTYDSVEKQKMSPENFERSSGQGKSVLFNPPIE
EFAVLYTTFQDGVGTRHFEGLHGPSIVITTKGNGFIKTGDLKLKAEPGFV
FFIAPGTEVDFIADDTDFTTYRAFVEPN
K. lactis SEC53, PMM:
>CAD21466 phosphomannomutase [Kluyveromyces
lactis].

(SEQ ID NO: 9)

MSVSEFSHKEQPTTLVLFDVDGTLTPARLTVSDEVRDILKRLREKVVIGF
VGGSDLSKQLEQLGSDVLDQFDYAFSENGLTAYRLGKELASQSFIEWIGE
EEYNKLAKFILQYLASIDLPKRRGTFLEFRNGMINVSPIGRNASTAERNE
FEQFDKEHQVRAKFVEALKKEFAHLSLTFSIGGQISFDVFPTGWDKTYCL
RHVEADGFKEIHFFGDKTYKGGNDYEIYVDDRTIGHSVESPADTVRILKE
LFDEL
human FUCT1 (GDP-fucose transporter):
>Q96A29 human GDP-fucose transporter FUCT1 GDF-
fucose transporter 1 (Solute carrier family 35
member C1) [Homo sapiens].

(SEQ ID NO: 10)

MNRAPLKRSRILHMALTGASDPSAEAEANGEKPFLLRALQIALVVSLYWV
TSISMVFLNKYLLDSPSLRLDTPIFVTFYQCLVTTLLCKGLSALAACCPG
AVDFPSLRLDLRVARSVLPLSVVFIGMITFNNLCLKYVGVAFYNVGRSLT
TVFNVLLSYLLLKQTTSFYALLTCGIIIGGFWLGVDQEGAEGTLSWLGTV
FGVLASLCVSLNAIYTTKVLPAVDGSIWRLTFYNNVNACILFLPLLLLLG
ELQALRDFAQLGSAHFWGMMTLGGLFGFAIGYVTGLQIKFTSPLTHNVSG
TAKACAQTVLAVLYYEETKSFLWWTSNNMVLGGSSAYTWVRGWEMKKTPE
EPSPKDSEKSAMGV
mouse SLC35a2 (UDP-galactose transporter):
>CAM24549 solute carrier family 35 (UDP-galactose
transporter) member 2 [Mus musculus].

(SEQ ID NO: 11)

MAAVGVGGSTAAAGAGAVSSGALEPGSTTAAHRRLKYISLAVLVVQNASL
ILSIRYARTLPGDRFFATTAVVMAEVLKGLTCLLLLFAQKRGNVKHLVLF
LHEAVLVQYVDTLKLAVPSLIYTLQNNLQYVAISNLPAATFQVTYQLKIL
TTALFSVLMLNRSLSRLQWASLLLLFTGVAIVQAQQAGGSGPRPLDQNPG
AGLAAVVASCLSSGFAGVYFEKILKGSSGSVWLRNLQLGLFGTALGLVGL
WWAEGTAVASQGFFFGYTPAVWGVVLNQAFGGLLVAVVVKYADNILKGFA
TSLSIVLSTVASIRLFGFHLDPLFALGAGLVIGAVYLYSLPRGAVKAIAS
ASASGPCIHQQPPGQPPPPQLSSRGDLTTEPFLPKLLTKVKGS
human UGT1 (UDP-galactose transporter):
>BAA12673 human UDP-galactose transporter UGT1UDP-
galactose translocator [Homo sapiens].

(SEQ ID NO: 12)

MAAVGAGGSTAAPGPGAVSAGALEPGTASAAHRRLKYISLAVLVVQNASL
ILSIRYARTLPGDRFFATTAVVMAEVLKGLTCLLLLFAQKRGNVKHLVLF
LHEAVLVQYVDTLKLAVPSLIYTLQNNLQYVAISNLPAATFQVTYQLKIL
TTALFSVLMLNRSLSRLQWASLLLLFTGVAIVQAQQAGGGGPRPLDQNPG
AGLAAVVASCLSSGFAGVYFEKILKGSSGSVWLRNLQLGLFGTALGLVGL
WWAEGTAVATRGFFFGYTPAVWGVVLNQAFGGLLVAVVVKYADNILKGFA
TSLSIVLSTVASIRLFGFHVDPLFALGAGLVIGAVYLYSLPRGAAKAIAS
ASASASGPCVHQQPPGQPPPPQLSSHRGDLITEPFLPKSVLVK
Schizosaccharomyces pombe GMS1 (UDP-galactose
transporter):
>P87041 S.pombe UDP-galactose transporter Gms1
UDP-galactose transporter [Schizosaccharomyces
pombe].

(SEQ ID NO: 13)

MAVKGDDVKWKGIPMKYIALVLLTVQNSALILTLNYSRIMPGYDDKRYFT
STAVLLNELIKLVVCFSVGYWQFRKNVGKEAKLRAFLPQIFGGDSWKLAI
PAFLYTCQNNLQYVAAGNLTAASFQVTYQLKILTTAIFSILLLHRRLGPM
KWFSLFLLTGGIAIVQLQNLNSDDQMSAGPMNPVTGFSAVLVACLISGLA
GVYFEKVLKDTNPSLWVRNVQLSFFSLFPCLFTILMKDYHNIAENGFFFG
YNSIVWLAILLQAGGGIIVALCVAFADNIMKNFSTSISIIISSLASVYLM
DFKISLTFLIGVMLVIAATFLYTKPESKPSPSRGTYIPMTTQDAAAKDVD
HKH
Saccharomyces cerevisiae HUT1 (UDP-galactose
transporter):
>NP_015080 S.cerevisiae UDP-galactosetransporter
Hut1 Hut1p [Saccharomyces cerevisiae].

(SEQ ID NO: 14)

MAGSTSSLVICAIGIYATFLTWALVQEPLATRTWPNSMGKFQFPNVISLI
QASVAMMMGYLYLNWKKVEYPPRKMIKDHWKQLMLISFTQSSSGPLATTS
LKHVDYLTYMLAKSCKMIPVLLVHLLLYRTPIASQKKVVALLVSLGVTIF
TIGGNDGKKLKRSFNESGNDNKLQGFGLLFSSLFLDGLTNATQDKLLKAN
KAKEKGKQTLITGAHLMFTLNLFVILWNILYFIVIDCKQWDNAVSVLTMD
PQVWGYLMLYSFCGAMGQCFIFYTLEQFGSLVLIMITVTRKNVSMILSII
VFGKSVRFQQWVGNFIVFGGITWEALNKKKANIPKAKSA
K. lactis GNT1 (MNN2-1) (N-acetylglucosaminyl-
transferase):
>AAD25740 alphaN-acetylglucosamine transferase
[Kluyveromyces lactis].

(SEQ ID NO: 15)

MAFGSRRKIKAILVAASAMVFISLLGTFGSDSVYEKIKTFDVSWGSNVSG
GLSSMLQKKKTVLYDPENIKQIPYSTIQKLYDHELESVTNIDWSQYAYVN
YVADKNYVCSSMIHFNRLHESGTQAKLVMLVAKELTELPEDDSVTRMLAQ
FKEISDNCIVKPVENIVLSQGSAQWMTSMTKLRVFGMVEYKRIVYFDSDS
IITRNMDELFFLPDYIQFAAPATYWFLNDNDLPQLIEDNKQIALANNQTA
ELTEIEDILQQKIDDSEDIYNFLPNLPKRLYPKSDNARIDSTDNTYFKYA
ATLMVIKPEQEMFERLEQEVLPKYLNTTNKYDMDLINIEFYDFNGTAEAQ
KKLYDQSPQSFKPSMLVLPFNQYTLLTKTIREKNRVKLLSNDMLGYETKK
PTDFRDASYYHFSDSPIGKPWKYKGLEDIPCNPGDSEEICNAWHSIFSNF
WDGRAKYCVA
K. lactis MNN2 (MNN2-2) (UDP-N-acetylglucosamine
transporter):
>XP_451673 MNN2_KLULA UDP-N-acetylglucosamine
transporter [Kluyveromyces lactis].

(SEQ ID NO: 16)

MSFVLILSLVFGGCCSNVISFEHMVQGSNINLGNIVTFTQFVSVTLIQLP
NALDFSHFPFRLRPRHIPLKIHMLAVFLFFTSSVANNSVFKFDISVPIHI
IIRCSGTTLTMIIGWAVCNKRYSKLQVQSAIIMTLGAIVASLYRDKEFSM
DSLKLNTDSVGMTQKSMFGIFVVLVATALMSLLSLLNEWTYNKCGKHWKE
TLFYSHFLALPLFMLGYTRLRDEFRDLLISSDSMDIPIVKLPIATKLFML
IANNVTQFICIKGVNMLASNTDALTLSVVLLVRKFVSLLLSVYIYKNVLS
VTAYLGTITVFLGAGLYSYGSVKTALPR
Schizosaccharomyces pombe GMA12:
>CAA83200 S.pombe alpha-1,2galactosyltransferase
Gma12 alpha-1,2-galactosyltransferase
[Schizosaccharomyces pombe].

(SEQ ID NO: 17)

MRFAPYLISAVVITTIILGGAWWTSAMDTKLQTKMKEIIDQHTSTWTPVV
SSVTSTQTDTLRVTISEVVSVTATLTETFTATPTVTSVVHALATTDPHPD
NSKIVILMGSNFQNDANSPLHPFAQSIIKNRREYAEREGYKFEFLDADAY
ASRVTGHLMPWVKVPMLQDTMKKYPDAEWIWWLDHDALVMNKDLNVVDHV
LKHDRLNTILTREAEYKSGAGIPADGFRTPKDQDAKDVHFIISQDFNGIN
AGSLFIRNSEVGRWIVDLWFEPLYLDHIQGYAEQQAFSHMVFYHPQVYKH
VGVVPLKAINAYDFDDNIWGYDDGDLCIHFAGCNYFKNCPEKFLKYAQIL
SSKQGSDWMSAQEKDHIQNLLKPSS
Schizosaccharomyces pombe PVG3:
>CAB58972 S.pombe beta-1,3galactosyltransferase
Pvg3 beta-1,3-galactosyltransferase (PMID
15173185) [Schizosaccharomyces pombe].

(SEQ ID NO: 18)

MFSNSKKKIFLYVLIAGVATFSFAFLVLNRLQAEEHSLAYVENLFLDFFI
KQNESLAHANDRPFKLYLGIFSQAKNVDRRNFLRTDYNEYIKEFAVNDTV
DVRFILGLPENEQELATIREEQRTYGDLAVLPIPENVDAGKSIVYFQTFL
EGYQPFPLFSELADNLIMPSTQFHGSFIYNQSIKTYELPGMKEFQDLGEP
KHDYDFIVKADDDSFLNLPRLFEMLKEHVGKSRFYFGRDCTRRELPTAVR
DFPYMCGFFYIVSPDMAYEVAKRRNIIIPFEDAQTGYSIYLSGNVKNAEF
SKCTLYDLILPNEGFNYRQSYLRIDAIAVHKLKSIPLLSTVSNWFKKMYE
HRANCSALIETERLSCLQATIPLPSLDV
human (1,2) fucosyltransferase FUT1:
>AAH74732 humanalpha-1,2fucosyltransferase
FUT1Fucosyltransferase 1 [Homo sapiens].

(SEQ ID NO: 19)

MWLRSHRQLCLAFLLVCVLSVIFFLHIHQDSFPHGLGLSILCPDRRLVTP
PVAIFCLPGTAMGPNASSSCPQHPASLSGTWTVYPNGRFGNQMGQYATLL
ALAQLNGRRAFILPAMHAALAPVFRITLPVLAPEVDSRTPWRELQLHDWM
SEEYADLRDPFLKLSGFPCSWTFFHHLREQIRREFTLHDHLREEAQSVLG
QLRLGRTGDRPRTFVGVHVRRGDYLQVMPQRWKGVVGDSAYLRQAMDWFR
ARHEAPVFVVTSNGMEWCKENIDTSQGDVTFAGDGQEATPWKDFALLTQC
NHTIMTIGTFGFWAAYLAGGDTVYLANFTLPDSEFLKIFKPEAAFLPEWV
GINADLSPLWTLAKP
human (1,2) fucosyltransferase FUT2:
>NP_001091107 human alpha-1,2fucosyltransferase
FUT2 fucosyltransferase 2 [Homo sapiens].

(SEQ ID NO: 20)

MLVVQMPFSFPMAHFILFVFTVSTIFHVQQRLAKIQAMWELPVQIPVLAS
TSKALGPSQLRGMWTINAIGRLGNQMGEYATLYALAKMNGRFAFIPAQMH
STLAPIFRITLPVLHSATASRIPWQNYHLNDWMEEEYRHIPGEYVRFTGY
PCSWTFYHHLRQEILQEFTLHDHVREEAQKFLRGLQVNGSRPGTFVGVHV
RRGDYVHVMPKVWKGVVADRRYLQQALDWFRARYSSLIFVVTSNGMAWCR
ENIDTSHGDVVFAGDGIEGSPAKDFALLTQCNHTIMTIGTFGIWAAYLTG
GDTIYLANYTLPDSPFLKIFKPEAAFLPEWTGIAADLSPLLKH
H. pylori futC:
>AAD29869 alpha-1,2-fucosyltransferase
[Helicobacter pylori].

(SEQ ID NO: 21)

MAFKVVQICGGLGNQMFQYAFAKSLQKHSNTPVLLDITSFDWSDRKMQLE
LFPINLPYASAKEIAIAKMQHLPKLVRDALKCMGFDRVSQEIVFEYEPEL
LKPSRLTYFYGYFQDPRYFDAISPLIKQTFTLPPPPENNKNNNKKEEEYH
RKLSLILAAKNSVFVHIRRGDYVGIGCQLGIDYQKKALEYMAKRVPNMEL
FVFCEDLEFTQNLDLGYPFMDMTTRNKEEEAYWDMLLMQSCQHGIIANST
YSWWAAYLIENPEKIIIGPKHWLFGHENILCKEWVKIESHFEVKSQKYNA

The invention provides for a method for producing yeast containing GDP-fucose comprising culturing the yeast harboring a recombinant nucleic acid molecule encoding GDP-mannose-4,6-dehydratase (GMD) and a recombinant nucleic acid molecule encoding GDP-L-fucose synthetase (GFS) under conditions in which the encoded GDP-mannose-4,6-dehydratase and the encoded GDP-L-fucose synthetase are expressed.

Also provided is a method for producing yeast that are competent for the Golgi import of both GDP-fucose and UDP-galactose comprising culturing the yeast that further harbors a nucleic acid molecule encoding a nucleotide sugar transporter under conditions in which the encoded nucleotide sugar transporter is expressed.

The invention also provides a method for producing yeast that lack terminal α(1,2) GlcNAc mannans comprising introducing the Kluyveromyces lactis yeast cell mnn2-1 or mnn2-2 mutation into the yeast cell that further harbors a nucleic acid molecule encoding a nucleotide sugar transporter.

Also provided is a method of producing yeast with α(1,2) galactose residues on surface polymannose, comprising culturing the yeast that further harbors a nucleic acid molecule encoding an α(1,2) galactosyltransferase under conditions in which the encoded α(1,2) glycosyltransferase is expressed.

The invention also provides a method of producing yeast with β(1,3) galactose linked to surface α(1,2) galactose residues comprising culturing the yeast that further harbors a nucleic acid molecule encoding a β(1,3) galactosyltransferase under conditions in which the β(1,3) glycosyltransferase is expressed.

A method of producing yeast with α(1,2) fucosylated glycans on its cell surface comprising is carried out by culturing the yeast that further harbors a nucleic acid molecule encoding a fucosyltransferase under conditions in which the fucosyltransferase is expressed.

The invention also provides for a method of preventing, treating, or reducing an infection, or the risk of developing an infection in a mammalian subject (human or animal) comprising administering a yeast cell that displays α(1,2) fucosylated glycans on its cell surface to a subject. The subject is infected with or at risk of becoming infected with a pathogen that binds to a fucosylated glycan. The glycans inhibit or reduce binding of a pathogen to the cells of the subject and thereby reduce infection. In one aspect, the infection is caused by a norovirus such as Norwalk-like virus, or strains of bacteria from genera including, but not limited to, Campylobacter (e.g. C. jejuni), Escherichia (e.g. E. coli), Salmonella (e.g. S. enterica) Vibrio (e.g. V. cholerae), or Helicobacter (e.g. H. pylori). The therapeutic compositions are useful not only for humans but also for prophylactic and therapeutic intervention to prevent and/or inhibit binding and infection of pathogens in livestock or companion animals such as pigs, horses, chickens, cows, sheep, goats, dogs, or cats. The formulations are suitable for administration to wild and domesticated animals.

Optionally, the invention features a vector, e.g., a vector containing a nucleic acid. The vector can further include one or more regulatory elements, e.g., a heterologous promoter or elements required for translation in yeast. The regulatory elements can be operably linked to a fusion protein in order to express the fusion protein. In yet another aspect, the invention features an isolated recombinant cell, e.g., a yeast cell containing an aforementioned nucleic acid molecule or vector. The nucleic acid sequence is optionally integrated into the genome. The sequence of an exemplary K. lactis expression vector is provided below; the vector includes two K. lactis promoter sequences “s2” and “s3” in the expression plasmid which possess strong promoter activity.

K. lactis expression vector pEKs2 3deltaU-fcl-gmd

(SEQ ID NO: 22)

tttaaacGCTTTTTCTTTCCAATTTTTTTTTTTTCGTCATTATAGAAATC
ATTACGACCGAGATTCCCGGGTAATAACTGATATAATTAAATTGAAGCTC
TAATTTGTGAGTTTAGTATACATGCATTTACTTATAATACAGTTTTTTAG
TTTTGCTGGCCGCATCTTCTCAAATATGCTTCCCAGCCTGCTTTTCTGTA
ACGTTCACCCTCTACCTTAGCATCCCTTCCCTTTGCAAATAGTCCTCTTC
CAACAATAATAATGTCAGATCCTGTAGAGACCACATCATCCACGGTTCTA
TACTGTTGACCCAATGCGTCTCCCTTGTCATCTAAACCCACACCGGGTGT
CATAATCAACCAATCGTAACCTTCATCTCTTCCACCCATGTCTCTTTGAG
CAATAAAGCCGATAACAAAATCTTTGTCGCTCTTCGCAATGTCAACAGTA
CCCTTAGTATATTCTCCAGTAGCTAGGGAGCCCTTGCATGACAATTCTGC
TAACATCAAAAGGCCTCTAGGTTCCTTTGTTACTTCTTCCGCCGCCTGCT
TCAAACCGCTAACAATACCTGGGCCCACCACACCGTGTGCATTCGTAATG
TCTGCCCATTCTGCTATTCTGTATACACCCGCAGAGTACTGCAATTTGAC
TGTATTACCAATGTCAGCAAATTTTCTGTCTTCGAAGAGTAAAAAATTGT
ACTTGGCGGATAATGCCTTTAGCGGCTTAACTGTGCCCTCCATGGAAAAA
TCAGTCAAGATATCCACATGTGTTTTTAGTAAACAAATTTTGGGACCTAA
TGCTTCAACTAACTCCAGTAATTCCTTGGTGGTACGAACATCCAATGAAG
CACACAAGTTTGTTTGCTTTTCGTGCATGATATTAAATAGCTTGGCAGCA
ACAGGACTAGGATGAGTAGCAGCACGTTCCTTATATGTAGCTTTCGACAT
GATTTATCTTCGTTTCCTGCAGGTTTTTGTTCTGTGCAGTTGGGTTAAGA
ATACTGGGCAATTTCATGTTTCTTCAACAgtcgaCTGTGCTCCTTCCTTC
GTTCTTCCTTCTGCTCGGAGATTACCGAATCAAAAAAATTTCAAACAAAC
CGGAATCAAAAAAAAGAACAAAAAAAAAAAAGATGAATTGAAAAGCgcGG
CCGtGCACAAACGAACGTCTCACTTAATCTTCTGTACTCTGAAGAGGAGT
GGGAAATACCAAGAAAAACATCAAACTCGAATGATTTTCCCAAACCCCTA
CCACAAGATATTCATCAGCTGCGAGATAGGCTGATCAGGAGCAAGCTCGT
ACGAGAAGAAACAAAATGACAAAAAAAATCCTATACTATATAGGTTACAA
ATAAAAAAGTATCAAAAATGAAGCCTGCATCTCTCAGGCAAATGGCATTC
TGACATCCTCTTGAGgatctCTCGAGTTTATGACTCCAGCGCGATCGCCA
CGTCGTAGCCGTGAGATTTCAGCAGAGAGTGTTTTTTCGCCGCTTCGAGG
TCATTAGCCACCATTTCAGACACCATCTCTCTGAGGGTGATTTCCGGTTT
CCAGCCCAGTTTTTCGTGCGCTTTGGTCGGGTCGCCGAGCAGCGTTTCAA
CTTCAGCCGGACGGAAGTAACGCGGGTCAACAGCGATAATCACATCACCC
GGTTTAACGCCCGGCGCGTCATGCCCGGTGACGCAAACCACAATGCCCTT
CTCTTCAACGCCCGTGCCTTCAAAGCGCAGTTTGATGCCCAGCTGTGCTG
CCGCCATTTCCACGAACTGACGCACGGAGTACTGAACGCCGGTCGCGATA
ACGAAATCTTCCGGCTGTTCCTGCTGCAGCATCATCCACTGCATTTTTAC
GTAGTCTTTGGCGTGGCCCCAGTCACGCAGGGAATCCATATTGCCGAGGT
ACAGGCACGACTCCAGCCCCTGGGCGATGTTGGCGATTGCGCGGGTGATT
TTGCGGGTAACGAAGGTTTCGCCGCGGCGCGGGGATTCATGGTTGAAGAG
AATTCCGTTACAGGCGTACATGCCGTAGGATTCACGGTAGTTAACGGTGA
TCCAGTAGGCGTACAGTTTGGCGACCGCATACGGAGATCGCGGGTAGAAC
GGCGTGGTCTCTTTCTGCGGAATTTCCTGCACCAGACCATACAGTTCAGA
GGTGGAAGCCTGATAGAAACGAGTTTTCTTTTCCAGACCGAGGAAGCGGA
TCGCCTCCAGCAGGCGCAGCGTACCCATCGCGTCGACGTCAGCGGTATAT
TCTGGTGACTCAAAAGAGACCGCAACGTGGCTCATTGCGCCCAGGTTGTA
CACTTCATCCGGCTGTACTTCACGCAAAATGCGCGTCAGGTTAGAGGTAT
CACTCAGGTCGCCATAATGCAGATGGAATTTCGGGTTGCAGGTGTGCGGA
TCCTGATAAATGTGATCCACGCGCTCGGTGTTGAATGACGATGCGCGACG
CTTAATACCATGCACCTCGTAACCTTTTTCCAGCAGAAACTCTGCCAGGT
AAGAACCGTCTTCTCCGGTTACACCGGTGATGAGAGCGACTTTTGACATT
TTTTTCAAGCTTactagtGGATCCAGATCAGCAGTAGCTCTCTGTGCCTC
TTTTCCTCTATCCTGTTTTATGCAAAATGTGCTATCGTAATGGTAAAATT
CAATGGCTTAAAGGTTGAAATTTTATTTCAAAAGATTCACAGCTTTTCCT
CTTTACAATGTAAAAATTTTTTTTTTCTTCTTTCGTGTATATTGAGAAGA
TTGACATGATGGTCAAGTTTATGACGAGATGAGATGCGATGAGGGGGAAA
AAGAGAGGATCTGCGGCCGtGCACAAACGAACGTCTCACTTAATCTTCTG
TACTCTGAAGAGGAGTGGGAAATACCAAGAAAAACATCAAACTCGAATGA
TTTTCCCAAACCCCTACCACAAGATATTCATCAGCTGCGAGATAGGCTGA
TCAGGAGCAAGCTCGTACGAGAAGAAACAAAATGACAAAAAAAATCCTAT
ACTATATAGGTTACAAATAAAAAAGTATCAAAAATGAAGCCTCCATCTCT
CAGGCAAATGGCATTCTGACATCCTCTTGAGgatctctcgagtttaCCCC
CGAAAGCGGTCTTGATTCTCAAGGAACCACTGGTAAGTGCTGGCAAGCCC
CGCTTCCAGTGAGATTTCGTGATACCAGCCAAGCTGATGCAGGCGCGTCA
CATCCAGCAGTTTGCGCGGCGTGCCATCCGGTTTGCTGGCATCAAAAACC
ACCCGGCCTTTGTAACCCACCACTTTGGCGATGGTTTGCGCCAGCTCGCG
GATAGTGCAGTCAACGCCCGTGCCGACGTTAATGTGCGACAACATCGGCT
GGGTGTTCTCCAGCCAGACTTCATCCGCCAGCTCCATGACATGAATGCTC
GCCGCCGCCATATCATCGACGTGCAGAAATTCGCGCATCGGTGTACCGCT
GCCCCATACCACCACGTCCGGCGCATTCTGTGCCGTCGCCTCGTGGAAGC
GACGCAGCAATGCTGGGATCACATGCGAATTACTCGGGTGGAAGTTGTCG
TGTGGCCCGTACAGGTTGGTCGGCATGACTGAGCGGTAATCGCGTCCGTA
CTGGCGGTTGTATGATTCGCACAGTTTGATCCCGGCGATTTTGGCAATAG
CATAAGGCTCGTTAGTCGGCTCCAGCGTGCCCTGCAACAACTCGCTTTCT
GCCATCGGCTGTTTTGCCAGTTTCGGGTAGATGCAGGACGATCCGAGAAA
CAGCAGTTTGTTCACGTCGTTCTGATGCGCGGCGTGAATGATGTTGCTCT
CAATCATCATGTTCTGGTAGATGAAATCCGCCGGATAGGTGTTGTTGGCA
ACAATGCCGCCCACTTTCGCCGCCGCCAGATAGACCTGGTCAATACGTTC
GCTGGCAAAGAAATCATGCACGGCGCGGCTGTCCAGCAGGTTCAGCTCGT
CGCGGGTGCGTAATACCAGTTCCACATCACCGCGCTGTTCGAGCTGCCGC
CTGATGGCGGAACCGACCATCCCGCGATGACCAGCAATAAAAACTCGTTG
TTTACTCATTTTTTTCAAGCTTactagtGGATCCAGATCGGACGGGAAAC
GGTGCTTTCTGGTAGATATGGCCGCAACCGAAATCTTAAGCAGTTACAGT
GGTAGTAGTTGTGTCTGGCTTACGGTTGTAGTATGTTGTTAGGCCAATTT
ATTTAGGAAGTGGGCTAGCTAACTTTATTGTTTGCTTAGATATATTTTTT
CAATTATTTTTCATTTTTTCATTTTTTTCATTTTTTTATTTTTTTATTTT
TTTATTTTTTTATAATTGGCTTCTTGTGGGATGTATAGTTTTTTTTATTT
TTCATACCGGATCTAATACTTGCAAACGACCGTACTTCAATCGTAGTGGT
TCTATATGTGCTATATTTGAAATCGAAGATATTTCTGCCGTTGCATCATC
TAGCCTTTTCTCGATAAGATCTGCGGCCgcATCTACACGAAGAGGGAAAA
TTGACTCCTGCCAGGAAACTTGACCGTACCACTCTTTTCCATACCGTTAG
AGATGTTTTACTTCATGATGGGTCTTACACCCGTCACAAAGGGGTCCGTT
GTTTGGACAACGGTAGCGTTAGAGTGAGCATACGGCTTAAGCACCGTCAA
TTAACCGCTACCTGGATAGAATTGGAACCCATTATTGAGAATGGCGAGGT
GAAAGACGTTATGTTCAAATTGTCCACCAGAGTACAATATACAGAGGAAA
ACGAGAACGAAACGCGCCCAGAATCTTCTTCAGAATGAGTGCATGTCAGA
CGCAGGCGTCGTATGCTTTTCATTAGTGATGCATGCCGTTGCAGCGTGCG
AATCGGCACGGTAATGATTCGAAATCTCTAATTATAATTACCTTCTGATA
TATAGATGAAAGACTATTTAATGAGAATATTGGACAGTACCGTTGGACTT
CGCTGACAGGTACATGGGCATTTTGGTTATTGGATGTAGAATGGTTGAAT
AAACGTGATTGTAAAATAGAGTTTGTAACTACGAATAATTAGTTTTTGAG
AAGTTTGGTGAATTTAATATTTGTATGAGGAAAGTAAATTTTAATACCTA
AATAAACAAAAATATATGGTACAGGAACGCGAGGCAACGCGCCGATACAG
GGTCAATGGGTACACGAGAGGGTGACACTAGGCGTAGAAAGTCATTAGTA
TAAAATACAGTGGTATATAGTAGATATTTAGTTTGTTTTCCTTTTCTTTT
TCTCCAAACGATATCAGACATTTGTCTGATAATGAAGCATTATCAGACAA
ATGTCTGATATCGTTTTTCAATAATAATATACATCATCACAAAACAAACA
AACATAGCATCGCAAGCCCCATCATGCCACCACCGTCCGCTGTGATCggc
gcgCCGCGGCTACTTTTCAATTCCCTATAGTGAGTCGTATTAAATTCGTA
ATCATGTCATAGCTGTTTCCTGTGTGAAATTGTTATCCGCTCACAATTCC
ACACAACATACGAGCCGGAAGCATAAAGTGTAAAGCCTGGGGTGCCTAAT
GAGTGAGCTAACTCACATTAATTGCGTTGCGCTCACTGCCCGCTTTCCAG
TCGGGAAACCTGTCGTGCCAGCTGCATTAATGAATCGGCCAACGCGCGGG
GAGAGGCCGTTTGCGTATTGGGCGCTCTTCCGCTTCCTCGCTCACTGACT
CGCTGCGCTCGGTCGTTCGGCTGCGGCGAGCGGTATCAGCTCACTCAAAG
GCGGTAATACGGTTATCCACAGAATCAGGGGATAACGCAGGAAAGAACAT
GTGAGCAAAAGGCCAGCAAAAGGCCAGGAACCGTAAAAAGGCCGCGTTGC
TGGCGTTTTTCCATAGGCTCCGCCCCCCTGACGAGCATCACAAAAATCGA
CGCTCAAGTCAGAGGTGGCGAAACCCGACAGGACTATAAAGATACCAGGC
GTTTCCCCCTGGAAGCTCCCTCGTGCGCTCTCCTGTTCCGACCCTGCCGC
TTACCGGATACCTGTCCGCCTTTCTCCCTTCGGGAAGCGTGGCGCTTTCT
CATAGCTCACGCTGTAGGTATCTCAGTTCGGTGTAGGTCGTTCGCTCCAA
GCTGGGCTGTGTGCACGAACCCCCCGTTCAGCCCGACCGCTGCGCCTTAT
CCGGTAACTATCGTCTTGAGTCCAACCCGGTAAGACACGACTTATCGCCA
CTGGCAGCAGCCACTGGTAACAGGATTAGCAGAGCGAGGTATGTAGGCGG
TGCTACAGAGTTCTTGAAGTGGTGGCCTAACTACGGCTACACTAGAAGGA
CAGTATTTGGTATCTGCGCTCTGCTGAAGCCAGTTACCTTCGGAAAAAGA
GTTGGTAGCTCTTGATCCGGCAAACAAACCACCGCTGGTAGCGGTGGTTT
TTTTGTTTGCAAGCAGCAGATTACGCGCAGAAAAAAAGGATCTCAAGAAG
ATCCTTTGATCTTTTCTACGGGGTCTGACGCTCAGTGGAACGAAAACTCA
CGTTAAGGGATTTTGGTCATGAGATTATCAAAAAGGATCTTCACCTAGAT
CCTTTTAAATTAAAAATGAAGTTTTAAATCAATCTAAAGTATATATGAGT
AAACTTGGTCTGACAGTTACCAATGCTTAATCAGTGAGCCACCTATCTCA
GCGATCTGTCTATTTCGTTCATCCATAGTTGCCTGACTCCCCGTCGTGTA
GATAACTACGATACGGGAGGGCTTACCATCTGGCCCCAGTGCTGCAATGA
TACCGCGAGACCCACGCTCACCGGCTCCAGATTTATCAGCAATAAACCAG
CCAGCCGGAAGGGCCGAGCGCAGAAGTGGTCCTGCAACTTTATCCGCCTC
CATCCAGTCTATTAATTGTTGCCGGGAAGCTAGAGTAAGTAGTTCGCCAG
TTAATAGTTTGCGCAACGTTGTTGCCATTGCTACAGGCATCGTGGTGTCA
CGCTCGTCGTTTGGTATGGCTTCATTCAGCTCCGGTTCCCAACGATCAAG
GCGAGTTACATGATCCCCCATGTTGTGCAAAAAAGCGGTTAGCTCCTTCG
GTCCTCCGATCGTTGTCAGAAGTAAGTTGGCCGCAGTGTTATCACTCATG
GTTATGGCAGCACTGCATAATTCTCTTACTGTCATGCCATCCGTAAGATG
CTTTTCTGTGACTGGTGAGTACTCAACCAAGTCATTCTGAGAATAGTGTA
TGCGGCGACCGAGTTGCTCTTGCCCGGCGTCAATACGGGATAATACCGCG
CCACATAGCAGAACTTTAAAAGTGCTCATCATTGGAAAACGTTCTTCGGG
GCGAAAACTCTCAAGGATCTTACCGCTGTTGAGATCCAGTTCGATGTAAC
CCACTCGTGCACCCAACTGATCTTCAGCATCTTTTACTTTCACCAGCGTT
TCTGGGTGAGCAAAAACAGGAAGGCAAAATGCCGCAAAAAAGGGAATAAG
GGCGACACGGAAATGTTGAATACTCATACTCTTCCTTTTTCAATATTATT
GAAGCATTTATCAGGGTTATTGTCTCATGAGCGGATACATATTTGAATGT
ATTTAGAAAAATAAACAAATAGGGGTTCCGCGCACATTTCCCCGAAAAGT
GCCACCTGACGCGCCCTGTAGCGGCGCATTAAGCGCGGCGGGTGTGGTGG
TTACGCGCAGCGTGACCGCTACACTTGCCAGCGCCCTAGCGCCCGCTCCT
TTCGCTTTCTTCCCTTCCTTTCTCGCCACGTTCGCCGGCTTTCCCCGTCA
AGCTCTAAATCGGGGGCTCCCTTTAGGGTTCCGATTTAGTGCTTTACGGC
ACCTCGACCCCAAAAAACTTGATTAGGGTGATGGTTCACGTAGTGGGCCA
TCGCCCTGATAGACGGTTTTTCGCCCTTTGACGTTGGAGTCCACGTTCTT
TAATAGTGGACTCTTGTTCCAAACTGGAACAACACTCAACCCTATCTCGG
TCTATTCTTTTGATTTATAAGGGATTTTGCCGATTTCGGCCTATTGGTTA
AAAAATGAGCTGATTTAACAAAAATTTAACGCGAATTTTAACAAAATATT
AACGCTTACAATTTCCATTCGCCATTCAGGCTGCGCAACTGTTGGCGAGG
GCGATCGGTGCGGGCCTCTTCGCTATTACGCCAGCTGGCGAAACGGGGAT
GTGCTGCAAGGCGATTAAGTTGGGTAACGCCAGGGTTTTCCCAGTCACGA
CGTTGTAAAACGACGGCCAGTGCCAAGCTCCCGCGG
K. lactis s2 Promoter

(SEQ ID NO: 23)

GATCTTATCGAGAAAAGGCTAGATGATGCAACGGCAGAAATATCTTCGAT
TTCAAATATAGCACATATAGAACCACTACGATTGAAGTACGGTCGTTTGC
AAGTATTAGATCCGGTATGAAAAATAAAAAAAACTATACATCCCACAAGA
AGCCAATTATAAAAAAATAAAAAAATAAAAAAATAAAAAAATGAAAAAAA
TGAAAAAATGAAAAATAATTGAAAAAATATATCTAAGCAAACAATAAAGT
TAGCTAGCCCACTTCCTAAATAAATTGGCCTAACAACATACTACAACCGT
AAGCCAGACACAACTACTACCACTGTAACTGCTTAAGATTTCGGTTGCGG
CCATATCTACCAGAAAGCACCGTTTCCCGTCCGATC
K. lactis s3 Promoter

(SEQ ID NO: 24)

GATCCTCTCTTTTTCCCCCTCATCGCATCTCATCTCGTCATAAACTTGAC
CATCATGTCAATCTTCTCAATATACACGAAAGAAGAAAAAAAAAATTTTT
ACATTGTAAAGAGGAAAAGCTGTGAATCTTTTGAAATAAAATTTCAACCT
TTAAGCCATTGAATTTTACCATTACGATAGCACATTTTGCATAAAACAGG
ATAGAGGAAAAGAGGCACAGAGAGCTACTGCTGATC

A “purified protein” refers to a protein that has been separated from other proteins, lipids, and nucleic acids with which it is naturally associated. Preferably, the protein constitutes at least 10, 20, 50 70, 80, 90, 95, 99-100% by dry weight of the purified preparation.

An “isolated nucleic acid” is a nucleic acid, the structure of which is not identical to that of any naturally occurring nucleic acid, or to that of any fragment of a naturally occurring genomic nucleic acid spanning more than three separate genes. The term covers, for example: (a) a DNA which is part of a naturally occurring genomic DNA molecule, but is not flanked by both of the nucleic acid sequences that flank that part of the molecule in the genome of the organism in which it naturally occurs; (b) a nucleic acid incorporated into a vector or into the genomic DNA of a prokaryote or eukaryote in a manner, such that the resulting molecule is not identical to any naturally occurring vector or genomic DNA; (c) a separate molecule such as a cDNA, a genomic-fragment, a fragment produced by polymerase chain reaction (PCR), or a restriction fragment; and (d) a recombinant nucleotide sequence that is part of a hybridgene, i.e., a gene encoding a fusion protein. Isolated nucleic acid molecules according to the present invention further include molecules produced synthetically, as well as any nucleic acids that have been altered chemically and/or that have modified backbones.

Although the phrase “nucleic acid molecule” primarily refers to the physical nucleic acid molecule and the phrase “nucleic acid sequence” refers to the sequence of the nucleotides the nucleic acid molecule, the two phrases can be used interchangeably.

The term “substantially pure” in reference to a given polypeptide means that the polypeptide is substantially free from other biological macromolecules. The substantially pure polypeptide is at least 75% (e.g., at least 80, 85, 95, or 99%) pure by dry weight. Purity can be measured by any appropriate standard method, for example, by column chromatography, polyacrylamide gel electrophoresis, or HPLC analysis.

A “heterologous promoter”, when operably linked to a nucleic acid sequence, refers to a promoter which is not naturally associated with the nucleic acid sequence.

The terms “express” and “over-express” are used to denote the fact that, in some cases, a cell useful in the method herein may inherently express some of the factor that it is to be genetically altered to produce, in which case the addition of the polynucleotide sequence results in over-expression of the factor. That is, more factor is expressed by the altered cell than would be, under the same conditions, by a wild type cell. Similarly, if the cell does not inherently express the factor that it is genetically altered to produce, the term used would be to merely “express” the factor since the wild type cell did not express the factor at all.

Engineered yeast cells displaying α(1,2) fucosylated glycans on the cell surface are potent pathogen adsorbents due to avidity effect (since the presentation these glycans are multivalent). The engineered yeast is provided as a food supplement—either as a live yeast or a killed (e.g. dried) yeast. The product is added to foods such as yogurts and kefir, or to weaning foods, or is included in a variety of other prepared foods, including cooked foods. In animal health applications the dried product, for example, is admixed with existing dried feedstuffs, e.g., in the form of kibble (e.g., for dogs) or pellets (e.g., for chickens, horses cattle). In the case of animal feedstuff, the yeast cells are combined with vegetable or meat (e.g., for dog food) or with corn and/or hay, soybeans, oats, wheat or other grains (e.g., for livestock).

Other features and advantages of the invention will be apparent from the following description of the preferred embodiments thereof, and from the claims. All references cited herein are hereby incorporated by reference.

### BRIEF DESCRIPTION OF THE DRAWINGS

FIG. 1 is a schematic illustration showing the synthetic pathway of the fucosyl oligosaccharides of human milk. Se and Le indicate synthesis by fucosyltransferases of the secretor and Lewis genes, respectively. The abbreviated biochemical name [with alternate biochemical structure in brackets] is given (histo-blood group antigen analog in parentheses).

FIG. 2 is a photograph illustrating the effect of the expression of α(1,2) fucosyl glycans in Chinese hamster ovary (CHO) cells transfected with the FUT2 gene (α 1,2 FUT⁺) on the binding of Campylobacter (top right) and V. cholerae (below), compared to bacterial binding in parental CHO cells carrying the plasmid vector only (top left, a 1,2 FUT⁻).

FIG. 3 is a bar graph demonstrating the inhibition of CV387 capsid binding by human milk from mothers of various blood group phenotypes.

FIG. 4 is a series of bar charts showing the concentration of fucosylated oligosaccharides in milk and the associated incidence of diarrhea in breastfed infants.

FIG. 5 is an overall scheme for the generation of cell-surface α(1,2) fucosylated K. lactis, and includes examples of the recombinant genes necessary to achieve expression.

FIG. 6 is a diagram illustrating the principle features of a recombinant DNA plasmid vector that, when transformed into K. lactis, directs the production of GDP-fucose in the cell.

FIG. 7 is a diagram illustrating the principle features of a recombinant DNA plasmid vector that, when transformed into K. lactis, directs the production of GDP-fucose and 2′-fucosyllactose in the cell.

FIG. 8 (Panel A) is a photograph of an immunoblot demonstrating expression of MYC-tagged E. coli fcl, MYC-tagged E. coli gmd and MYC-tagged H. pylori futC in K. lactis cells transformed with plasmid vectors containing the respective recombinant genes. Both single gene expression and combined two and three gene co-expression is demonstrated. (Panel B) is a photograph of a thin layer chromatogram of K. lactis cell extracts from cells co-expressing futC, fcl and gmd and grown in the presence of lactose. The TLC demonstrates the production of 2′-fucosyllactose.

FIG. 9 is a scheme illustrating the pathways to GDP-mannose synthesis in yeasts.

FIG. 10 is a diagram illustrating the principle features of recombinant DNA plasmid vectors that, when transformed into K. lactis, direct the over-production of native or MYC-tagged versions of K. lactis phosphomannose isomerase (PMI, KLLA0D13728g) K. lactis phosphomannomutase (PMM, SEC53) or K. lactis GDP-mannose pyrophosphorylase (PSA1).

FIG. 11 is a photograph of an immunoblot demonstrating over-expression of MYC-tagged K. lactis PMI, MYC-tagged K. lactis PMM and MYC-tagged K. lactis PSA1 in K. lactis cells transformed with plasmid vectors containing the respective recombinant genes.

FIG. 12 is a diagram (panel A) illustrating the K. lactis genome surrounding the GNT1 (MNN2-1) locus, encoding N-acetylglucosaminyltransferase. Also shown (panel B) is a diagram illustrating the principle features of pINT-1, a plasmid vector that does not replicate in K. lactis and that was designed to generate a specific 1000 bp deletion in the GNT1 gene by homologous recombination.

FIG. 13 is a diagram illustrating specific deletion using homologous recombination (in two steps) of most of the coding region (i.e. precisely 1000 bp, starting at the initiator ATG codon) of K. lactis GNT1 (MNN2-1), encoding N-acetylglucosaminyltransferase.

FIG. 14 (panel A) is a plot of a FACS run performed on a pool of 5FOA resistant K. lactis cells (stained with FITC labeled Griffonia simplicifonia lectin II, GSII) that were spontaneously derived from a prior growth of URA⁺SwaI-cleaved pINT-1 co-integrants (see FIG. 13) under non-selective conditions. The low fluorescence peak on the left are cells which resolved the co-integrant by deleting GNT1, the high fluorescence peak on the right are cells which resolved the co-integrant by reverting back to wild-type. (panel B) is photograph of a stained agarose gel performed on PCR reactions using primers on either side of the specific deletion in GNT1. PCR products derived from the 5FOA resistant candidates are ˜1 kb shorter than those derived from wild-type cells, consistent with the designed deletion of 1000 bp.

FIG. 15 is a photograph of an immunoblot demonstrating expression of MYC-tagged E. coli fcl, MYC-tagged E. coli gmd, MYC-tagged H. pylori futC, MYC-tagged S. pombe GMA12, MYC-tagged S. pombe PVG3, human FUT1 and human FUCT1 in K. lactis cells transformed with plasmid vectors containing the respective recombinant genes.

### DETAILED DESCRIPTION OF THE INVENTION

Adherence of pathogens to host cells is the obligatory first step in infection and is frequently mediated by specific molecular interactions. For example, Campylobacter species and Norwalk virus, the leading bacterial and viral causes of human infectious diarrhea, both adhere to gut epithelial surfaces through binding to specific sugars (i.e. α(1,2) fucosylated glycans) that are elaborated on the surface of intestinal cells. These same α(1,2) fucosylated glycans are also found in soluble forms in human breast milk, and recent work has demonstrated the critical role of these molecules, naturally provided by the mother to her baby, in the protection of infants from gut infections. For instance, there is a much higher risk of developing gut infections in infants: 1). at weaning, when protective factors found in mother's milk are reduced or removed from the child's diet, or 2). when infants are fed on conventional baby formula, which does not contain α(1,2) fucosylated glycans, or 3). for infants whose mothers are genetically incapable of providing sufficient α(1,2) fucosylated glycans in their milk.

The α(1,2) fucosylated glycans that are abundant in human breast milk effectively prevent binding of numerous important gut pathogens, both in vitro and in vivo, (such as Norwalk-like virus, or strains of bacteria from genera such as Campylobacter (e.g. C. jejuni), Escherichia (e.g. E. coli), Salmonella (e.g. S. enterica) and Vibrio (e.g. V. cholerae)), and thus prevent infection by these organisms. Thus, soluble α(1,2) fucosylated glycans present in human breast milk represent a class of natural anti-infective agents that provide an important layer of innate prophylactic protection to young infants. However, the production of α(1,2) fucosylated glycans as anti-infective agents in sufficient quantities to impact global diarrhea incidence remains a significant challenge. Chemical syntheses are possible, but are limited by stereo-specificity issues, product impurities, and high overall cost. In vitro enzymatic syntheses are also possible but are limited by a requirement for expensive nucleotide-sugar precursors. Accordingly, there is a pressing need for new strategies for α(1,2) fucosylated glycan-mediated inhibition of pathogen binding to gut epithelia.

### Travelers' Diarrhea

Travelers' diarrhea is a very common illness affecting travelers. It is estimated that between 20-50% of international travelers develop the condition every year, representing an annual incidence of greater than 10 million cases. Although the disease is typically self-limiting (90% of cases resolve without treatment within one week), symptoms are unpleasant and disruptive, and international travelers, particularly to high-risk destinations such as the developing countries of Latin America, Africa, the Middle East and Asia, are advised to take dietary precautions while traveling to reduce their risk of contracting the illness. Moreover more than 10% of individuals who have recovered from travelers' diarrhea progress to inflammatory bowel syndrome (IBS), a chronic debilitating condition with no good current treatment options. Eighty percent (80%) of travelers' diarrhea cases are attributed to infections by enterotoxigenic E. coli, and are usually contracted by ingestion of fecally contaminated food or water. Many of the symptoms induced by enterotoxigenic E. coli are caused by small heat-stable peptide toxins secreted by the organism, and are mediated through toxin binding to an intestinal cell-surface receptor, the guanylin cyclase receptor. α(1,2) fucosylated milk-derived glycans inhibit this specific toxin binding and protect against enterotoxigenic E. coli infections.

Current treatments for travelers' diarrhea are mostly palliative, OTC formulations of gut anti-motility agents such as loperamide (Imodium®) or diphenoxylate (Lomotil®) can provide symptomatic relief, but conversely can also delay pathogen clearance. Bismuth subsalicylate (Pepto-Bismol®) can be effective in alleviating symptoms, but is not an agent recommended for use by children under the age of 12, by pregnant women, or by people who are allergic to aspirin. Prophylaxis, other than by the unacceptable prophylactic use of antibiotics, is currently not an option against travelers' diarrhea. Diarrhea is a long-standing problem for the military and continues to be a dominant medical concern in deployed units. The compositions and methods described herein are useful in military-based applications.

### Probiotics

Probiotics are dietary supplements containing beneficial bacteria or yeasts. The World Health Organization characterizes probiotics as live microorganisms which when administered in adequate amounts confer a health benefit on the host.

The invention provides probiotic yeast strains expressing α(1,2) fucosylated glycans on their cell surface that act as “decoys”, which adsorb pathogenic organisms on their cell surface with high efficiency (due to avidity effects). The strains are provided in foods or beverages such as yogurt, kefir, cultured drinks, or infant formula which are marketed as “probiotics”. Alternatively, the microorganisms are provided dry, e.g., lyophilized, together with instructions for reconstitution by mixing with a liquid such as water, juice, or a dairy product.

The invention provides glycans representing a novel class of prophylactic and therapeutic agents. These agents are inexpensive to produce in large quantities, stable at room temperature, allow for large-scale storage, oral administration, and are safe and well tolerated. These glycans inhibit infection by a broad spectrum of pathogens, including but not limited to Campylobacter jejuni, caliciviruses (noroviruses, including Norwalk Virus), pathogenic vibrios, and certain diarrheagenic Escherichia and Salmonella strains (Chessa, D, Winter, M G, Jakomin, M and Bäumler, A J, Mol Microbiol 71:4, 864-75 (2009)).

### Fucosyl Glycans are Receptors for Pathogens

Attachment to the gastrointestinal mucosa is the essential first step in enteric infection. Cells express glycans on their surface to communicate with the external milieu, but these glycans are also used by pathogens for the process of host cell recognition and binding. Among cell surface glycans, those with fucosylated moieties are heavily expressed in intestinal mucosa and are used as target receptors for a number of enteric pathogens, including Campylobacter species, Vibrio cholerae, some diarrheagenic E. coli, human caliciviruses (noroviruses), Helicobacter pylori, and others (Ruiz-Palacios G. M. et al., 2003 J Biol Chem, 278:14112-14120; Marionneau S. et al., 2002 Gastroenterology, 122:1967-1977; Glass R. I. et al., 1985 Am J Epidemiol, 121:791-796; Barua D. and Paguio A. S., 1977 Ann Hum Biol, 4:489-492; Ever D. et al., 1998 Science, 279:373-377; Newburg D. S. et al., 1990 J Infect Dis, 162:1075-1080; Huang P. et al., 2003 J Infect Dis, 188:19-31). For example, children who are homozygous recessive for the “secretor” α(1,2)-fucosyltransferase gene (FUT2) under-express α(1,2)-linked glycans on their intestinal mucosal surface and are naturally resistant to diarrhea; those with no such mutation in their FUT2 alleles (i.e., homozygous dominant for FUT2) express the highest level of these fucosylated receptors and have the highest risk of diarrheal disease. Thus, cell surface glycan receptors are critical determinants of pathogenicity for many enteric and other pathogens.

### Human Milk Glycans

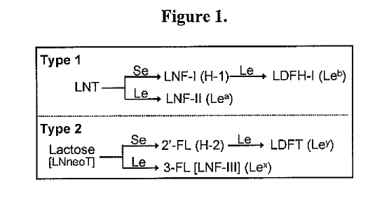

Human milk glycans, which comprise both unbound oligosaccharides and their glycoconjugates, play a significant role in the protection and development of the infant gastrointestinal (GI) tract. Milk oligosaccharides found in various mammals differ greatly, and composition in humans is unique (Hamosh M., 2001 Pediatr Clin North Am, 48:69-86; Newburg D. S., 2001 Adv Exp Med Biol, 501:3-10). Moreover, glycan levels in human milk change throughout lactation and also vary widely among individuals (Morrow A. L. et al., 2004 J Pediatr, 145:297-303; Chaturvedi P et al., 2001 Glycobiology, 11:365-372). Approximately 200 distinct human milk oligosaccharides have been identified and combinations of simple epitopes are responsible for this diversity (Newburg D. S., 1999 Curr Med Chem, 6:117-127; Ninonuevo M. et al., 2006 J Agric Food Chem, 54:7471-74801). Human milk oligosaccharides are composed of 5 monosaccharides: D-glucose (Glc), D-galactose (Gal), N-acetylglucosamine (GlcNAc), L-fucose (Fuc), and sialic acid (N-acetyl neuraminic acid, Neu5Ac, NANA). Human milk oligosaccharides are usually divided into two groups according to their chemical structures: neutral compounds containing Glc, Gal, GlcNAc, and Fuc, linked to a lactose (Galβ1-4Glc) core, and acidic compounds including the same sugars, and often the same core structures, plus NANA (Charlwood J. et al., 1999 Anal Biochem, 273:261-277; Martin-Sosa et al., 2003 J Dairy Sci, 86:52-59; Parkkinen J. and Finne J., 1987 Methods Enzymol, 138:289-300; Shen Z. et al., 2001 J Chromatogr A, 921:315-321). Approximately 70-80% of oligosaccharides in human milk are fucosylated. The majority of fucosylated oligosaccharides in most individuals contain one or more α(1,2)-linked fucoses, which are synthesized by a fucosyltransferase encoded by the FUT2 gene. About one-third of human milk fucosyl oligosaccharides include one or more α(1,3) or α(1,4)-linked fucoses, synthesized by fucosyltransferases encoded by the Lewis gene (FUT3) family (Le Pendu, 2004 Adv Exp Med Biol, 554:135-143). Together, these fucosyltransferases produce six major epitopes (FIG. 1; Type I and Type II pathways begin with different precursor molecules). In most mothers, the dominant milk oligosaccharides are 2′-fucosyllactose (2′-FL) and lacto-N-fucopentaose I (LNF-I). However, 20 to 25% of individuals of European, African and Asian, and 2-3% of Mexican ancestry are non-secretors, i.e., they lack an active FUT2 allele, and thus entirely lack α(1,2)-fucosyl glycans in their milk. These individuals have lower amounts of total oligosaccharide and a dominance of oligosaccharides containing only α(1,3) and α(1,4)-linked fucose.

Human Milk Glycans Inhibit Binding of Enteropathogens to their Receptors

Human milk glycans have structural homology to cell receptors for enteropathogens and function as receptor decoys. For example, pathogenic strains of Campylobacter bind specifically to glycans containing H-2, i.e., 2′-fucosyl-N-acetyllactosamine or 2′-fucosyllactose (2′FL); Campylobacter binding and infectivity are inhibited by 2′FL and other glycans containing this H-2 epitope. Similarly, some diarrheagenic E. coli pathogens are strongly inhibited in vivo by human milk oligosaccharides containing 2-linked fucose moieties. Several major strains of human caliciviruses, especially the noroviruses, also bind to 2-linked fucosylated glycans, and this binding is inhibited by human milk 2-linked fucosylated glycans. Consumption of human milk that has high levels of these 2-linked fucosyloligosaccharides was associated with lower risk of norovirus, Campylobacter, ST of E. coli-associated diarrhea, and moderate-to-severe diarrhea of all causes in a Mexican cohort of breastfeeding children (Newburg D. S. et al., 2004 Glycobiology, 14:253-263; Newburg D. S. et al., 1998 Lancet, 351:1160-1164).

### Therapeutic Glycans

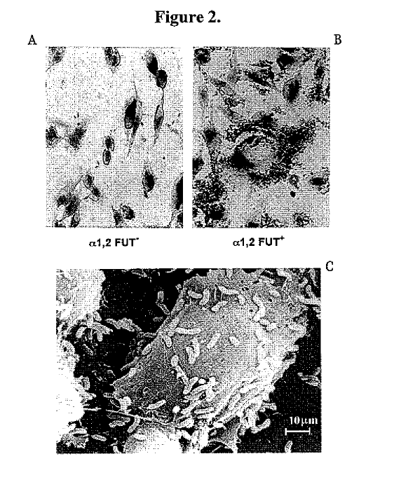

The human milk glycans represent a new class of powerful antimicrobial agents (Newburg D. S. et al., 2005 Annu Rev Nutr, 25:37-58; Sharon N. and Ofek I., 2000 Glycoconj 3, 17:659-664), and the data indicate that they do not induce emergent drug-resistance. Glycan structures isolated from human milk were found to inhibit infection by specific pathogens in vitro and in vivo. Some pathogens (e.g., C. jejuni, Vibrio cholerae) are inhibited by a specific fucosylated epitopes in the monomeric form, i.e., the free oligosaccharide, although inhibition is stronger with more complex forms. Other pathogens (e.g., noroviruses) are inhibited only by these epitopes when they are anchored to a macromolecule, as the polyvalent (multiple copies of the same epitope on a macromolecule) or multivalent (combinations of epitopes on a macromolecule) forms. In general, polyvalent and multivalent expression of oligosaccharides is accompanied by an increase in binding avidity to specific lectins. These observations indicate a need for multiple epitopes (as found in human milk) in a formulation that is intended to inhibit the multiplicity of pathogens found in a free-living population. However, 1) most human milk protective glycans contain in common only six neutral fucosylated glycan epitopes, the Lewis epitopes, which inhibit the most common major families of enteric pathogens (FIGS. 1 and 2) human milk contains only a few macromolecules that bind strongly to pathogens, and principal among them is the mucin expressed from the MUC1 gene. For example, recombinant fucosylated mucin inhibits binding by H. pylori.

### Limitations of Existing Synthesis Methods

While a wealth of published studies suggest that human milk glycans could be used as a novel class of antimicrobial anti-adhesion agents, the difficulty and expense of producing adequate quantities of these agents of a quality suitable for human consumption has limited their full-scale testing and perceived utility. Prior to the invention, there was a need for a suitable method for producing the appropriate glycans in sufficient quantities at reasonable cost. Synthetic approaches for glycan synthesis have been attempted. Novel chemical approaches can synthesize oligosaccharides, but reactants for these methods are expensive and potentially toxic. Enzymes expressed from engineered organisms provide a precise and efficient synthesis, but the high cost of the reactants, especially the sugar nucleotides, limits their utility for low-cost, large-scale production. Bacteria have been genetically engineered to express the glycosyltransferases needed to synthesize oligosaccharides from the bacteria's innate pool of nucleotide sugars. In one such example, all enzymes essential for oligosaccharide synthesis, including glycosyltransferases and the enzymes that comprise the nucleotide sugar regeneration pathway, were built into a single plasmid and expressed within one E. coli “superbug” (Chen X. et al., 2001 J Am Chem Soc, 123:8866-8867); oligosaccharides were synthesized on a g/L scale. However, bacteria produce cellular components, such as lipopolysaccharide and peptidoglycan, that are powerful antigens and toxins to humans thereby causing undesirable and dangerous side effects. Therefore, efforts were undertaken to develop cheaper and safer ways to manufacture human milk glycans.

### α(1,2) Fucosylated Glycans

α(1,2) fucosylated glycans present in human breast milk competitively inhibit the adherence of major pathogens to gut epithelia, and represent a class of anti-infective agents that provide prophylactic protection to young infants and adults against infection and/or colonization by gut pathogens. In addition to at risk infant populations, there exist several other additional large populations at risk for diarrheal disease (e.g. the elderly, international travelers, and military troops), who could benefit from prophylactic administration of these agents, were they readily available. However, as stated above, prior to the invention described herein, no current commercially feasible synthetic source of human breast milk-derived α(1,2) fucosylated glycans existed, and existing dairy products based on cow's or goat's milk are missing this class of molecules.

Prior to the invention, the production of α(1,2) fucosylated glycans as anti-infective agents in sufficient quantities to impact global diarrhea incidence was a significant challenge. Chemical syntheses are possible, but are limited by stereo-specificity issues, product impurities, and high overall cost (Flowers H. M., 1978 Methods Enzymol, 50:93-121; Seeberger P. H., 2003 Chem Commun (Camb), 1115-1121; Koeller K. M. and Wong C. H., 2000 Chem Rev, 100:4465-4494). In vitro enzymatic syntheses are also possible, but are limited by a requirement for expensive nucleotide-sugar precursors.

The invention describes less expensive ways of manufacturing these molecules in bulk as anti-infective agents. The invention also provides these agents as nutritional supplements and/or as therapeutics not only for at-risk infants (e.g., non-breast fed infants, partially breast-fed infants, breast-fed infants of mothers genetically incapable of providing sufficient protective glycans in their milk, weaning infants, infants in high risk environments for infectious diarrhea such as daycare centers, or locations with compromised water sanitation), but also for susceptible adults (e.g., the elderly, particularly in nursing home environments, for travelers, for all adults in high risk environments such as locations with compromised water sanitation). The invention also provides these agents as nutritional supplements or feedstuff additives for animals (e.g., pigs, cows, chickens) to reduce the risk of infectious diarrhea by organisms utilizing fucosylated glycans for pathogenesis (e.g., F18-fimbriated enterotoxigenic E. coli in pigs (Snoeck, V, Verdonck, F, Cox, E and Goddeeris, B M, Vet Microbiol 100:3-4, 241-6 (2004))

Glycan synthetic pathways were engineered in the common edible dairy yeast Kluyveromyces lactis through a combination of endogenous gene manipulation and the introduction of heterologous genes encoding desired activities. K. lactis was engineered to synthesize the key precursor sugar, GDP-fucose (optimizing the production of this nucleotide sugar and minimizing the impact of this on yeast cell wall synthesis by boosting the cellular GDP-mannose pool), the ability to transport both GDP-fucose and UDP-galactose to the Golgi, and the ability to elaborate α(1,2) fucosylated glycans on the cell surface. Yields of surface-bound α(1,2) fucosylated glycans were optimized. The engineered yeast was evaluated for pathogen binding in vitro and for use as a probiotic in an in vivo animal model of Campylobacter or Vibrio infection. By inhibiting pathogen adherence to epithelia, the engineered yeast is used to treat and/or prevent infection by various infections agents, e.g., by inhibiting infectious agents associated with various disorders such as enteric, pulmonary (airway), and urinary tract disorders. The surface α(1,2) fucosylated probiotic K. lactis targets both Norwalk-like viruses and C. jejuni, as well as other fucose-binding pathogens, including but not limited to pathogenic Vibrios, Helicobacter, Pseudomonas aeruginosa, Streptococcus pneumoniae, and certain diarrheagenic Escherichia and Salmonella strains.

### Selection of Expression Host

Kluyveromyces lactis was selected to display α(1,2) fucosylated glycoproteins for a variety of reasons. First, the organism is eukaryotic and possesses intrinsic mechanisms for glycan production and secretion. The K. lactis genome has been completely sequenced, and the organism is benign and easily fermented on simple media. Moreover, since K. lactis is established in the human diet in various cheeses, appropriately engineered forms of the organism or products produced by the organism may qualify for GRAS status (Generally Recognized as Safe) with the Food and Drug Administration (Leclercq-Perlat M. N. et al., 2004 J Dairy Res, 71:346-354; Fadda, M. E. et al., 2001 Int J Food Microbiol, 69:153-156; Prillinger, H. et al., 1999 Antonie Van Leeuwenhoek, 75:267-283).

Although it is well established that the wide use of conventional anti-bacterial agents and antibiotics leads to the development of resistant strains, this problem is avoided with the fucosylated glycan adherence inhibitors (an advantage over conventional antibiotic approaches). Antibacterial agents typically act directly on the growth and/or viability of target organisms and impose severe selective pressures that provide opportunity for the development of resistance. Anti-adherence anti-infective agents do not exert this direct selective pressure, instead they render the pathogen's environmental niche unavailable. Thus the compositions described herein do not drive the development of resistance, because pathogens have not evolved mechanisms to circumvent the natural anti-infective glycans present in mother's milk.

### Compositions Comprising Engineered Probiotic Yeast

The engineered probiotic yeast, e.g., K laths that displays α(1,2) fucosylated glycans on its cell surface, are administered as an over-the-counter food, beverage, or nutraceutical product or as a pharmaceutical composition containing the engineered yeast and a pharmaceutically acceptable carrier, e.g., phosphate buffered saline solution, mixtures of ethanol in water, water and emulsions such as an oil/water or water/oil emulsion, as well as various wetting agents or excipients. The engineered probiotic yeast are combined with materials that do not produce an adverse, allergic or otherwise unwanted reaction when administered to a patient. The carriers or mediums used include solvents, dispersants, coatings, absorption promoting agents, controlled release agents, and one or more inert excipients (which include starches, polyols, granulating agents, microcrystalline cellulose, diluents, lubricants, binders, disintegrating agents, and the like), etc. If desired, tablet dosages of the disclosed compositions are coated by standard aqueous ornonaqueous techniques.

The engineered probiotic yeast are administered orally, e.g., as a tablet containing a predetermined amount of the probiotic yeast, pellet, gel, paste, syrup, bolus, electuary, slurry, capsule, powder, granules, as a solution or a suspension in an aqueous liquid or a non-aqueous liquid; as an oil-in-water liquid emulsion or a water-in-oil liquid emulsion, or in some other form. Orally administered compositions optionally include binders, lubricants, inert diluents, lubricating, surface active or dispersing agents, flavoring agents, and humectants. Orally administered formulations such as tablets may optionally be coated or scored and may be formulated so as to provide sustained, delayed or controlled release of the probiotic yeast. In another aspect, the agents of the invention are administered by rectal suppository, aerosol tube, naso-gastric tube, direct infusion into the GI tract or stomach or parenterally.

Pharmaceutical compositions containing probiotic yeast can also include therapeutic agents such as antiviral agents, antibiotics, probiotics, analgesics, and anti-inflammatory agents. The proper dosage is determined by one of ordinary skill in the art and depends upon such factors as, for example, the patient's immune status, body weight and age. In some cases, the dosage is at a concentration similar to that found for similar oligosaccharides present in human breast milk.

The probiotic yeast are added to other compositions. For example, they are added to an infant formula, a nutritional composition, a rehydration solution, a dietary maintenance or supplement for elderly individuals or immunocompromised individuals. The probiotic yeast is included in compositions that include macronutrients such as edible fats, carbohydrates and proteins. Edible fats include, for example, coconut oil, soy oil and monoglycerides and diglycerides. Carbohydrates include, for example, glucose, edible lactose and hydrolyzed cornstarch. Protein sources include, for example, protein source may be, for example, soy protein, whey, and skim milk. Compositions, including nutritional compositions, containing the probiotic yeast can also include vitamins and minerals (e.g., calcium, phosphorus, potassium, sodium, chloride, magnesium, manganese, iron, copper, zinc, selenium, iodine, and Vitamins A, E, D, C, and B complex).

The engineered probiotic yeast displaying α(1,2) fucosylated glycans on the cell surface is delivered as a live culture, or alternatively as a preparation killed or substantially reduced in viability through treatments (e.g. drying) consistent with maintaining pathogen-binding activity for cell surface fucosylated glycans. Reduced viability forms of probiotic yeast are produced using know methods, e.g., flash-dried probiotic yeast is used as a convenient human food or animal feedstuff additive.

### Example 1

### Heterogeneity of Glycan Expression in Infants Defines their Risk of Diarrhea

In children, the Lewis histo-blood group antigens in the gastrointestinal tract are thought to serve as primary receptors for several major enteropathogens. Genetic polymorphisms of fucosyltransferases in the infant underlie heterogeneous fucose expression in gut and other tissues, reflected as differences in expression of the Lewis blood group phenotype in erythrocytes and saliva, and especially as Lewis genotype, which is more precise. If binding to cell surface fucosylated glycans on the intestinal mucosa is the essential first step of pathogenesis, the risk of diarrheal infections in the infant should be related to heterogeneous expression of fucosylglycans (Marionneau S. et al., 2001 Biochimie, 83:565-573).

A cohort of 297 Mexican infants was tested and followed from birth to 2 yr of age (Noguera-Obenza M. et al., 2003 Emerg Infect Dis, 9:545-551). The relative risk of diarrhea varied by blood type (Table 1). Children expressing fucose primarily as the Fuc α(1,2) epitope (0, Lewis a-b-) had the highest risk of diarrhea, 3.0 cases/child-year. Those expressing fucose primarily as Fuc α(1,4) (0, Lewis a+b-), but lacking Fuc α(1,2) epitope, had the lowest risk, 1.5 cases/child-year (P=0.002). The A and B blood group types (all express the Fuc α(1,2) epitope, but with an additional sugar at the terminus) had intermediate risks of diarrhea; the extra sugars may sterically hinder access to the active epitope, and the production of A and B glycans may deplete the amount of glycans with the more active Fuc α(1,2) epitope.

TABLE 1
Risk of diarrhea by Lewis ABO (H) blood type of humans

| ABO (H) | | All Diarrhea | | |
|---|---|---|---|---|
| Lewis | | | RR | P- |
| Group | | IR | (95% Cl) | Value |
| O^{a-b-} (N = 37) | 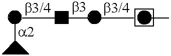 | 3.0 | 1.00 | |
| O^{a-b+} (N = 191) | 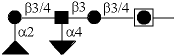 | 2.5 | 0.84 (0.72- 0.99) | 0.04 |
| O^{a-b+} (N = 55) B^{a-b+} | 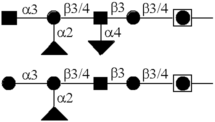 | 2.2 | 0.74 (0.61- 0.91) | 0.003 |
| A^{a-b-} (N = 7) B^{a-b-} | 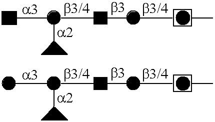 | 1.8 | 0.62 (0.29- 0.79) | 0.001 |
| O^{a+b-} (N = 7) | 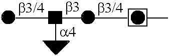 | 1.5 | 0.50 (0.40- 0.92) | 0.01 |

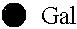

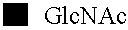

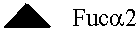

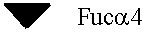

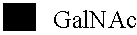

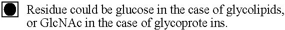

IR, Incidence Rates; RR. Relative Risk; CI, Confidence Interv

The differences in diarrhea susceptibility among blood group types indicate that human variation in glycan expression, specifically regarding expression of α(1,2)-linked fucose, is reflected in differences in pathogen susceptibility. Cell surface receptors containing α(1,2)-linked fucose are major determinants of pathogenicity of many enteric pathogens in human populations, because on the specificity of enteropathogen binding to their glycan receptors is an essential primary step in pathogenesis. Lewis blood group phenotypes and genotypes are powerful predictors of diarrhea in infants, with specific expression patterns rendering some children intrinsically more susceptible to these major enteropathogens.

### Example 2

### Specific Fucosyl Glycans Inhibit Specific Pathogens

### Glycan Inhibition of Campylobacter

Campylobacter jejuni binding to HEp2 cells is inhibited by fucosylated carbohydrate moieties containing the H(O) blood group epitope (Fucα(1,2)Galβ(1,4)GlcNAc) (Ruiz-Palacios 2003). With nitrocellulose-immobilized neoglycoproteins (high-molecular-weight synthetic glycans), Campylobacter has high avidity for the H-2 antigen, and this specificity was confirmed by inhibition with monoclonal antibodies. C. jejuni, which normally does not bind to Chinese hamster ovary (CHO) cells, bound avidly when the cells were transfected with a human α(1,2)-fucosyltransferase gene that caused overexpression of H-2 antigen (FIG. 2).

This binding was inhibited by ligands that bind to H-2, e.g., UEA I, Lotus lectins and anti-H-2 mAbs, or by H-2 soluble mimetics, e.g., H-2 neoglycoproteins, human milk fucosylated oligosaccharides and 2′FL, which compete with cell receptors. Thus, H-2 antigen was established as the host cell surface antigen that is the determinant of susceptibility to Campylobacter infection.

The relevance of inhibition of binding to the H-2 epitope in protection from disease was assessed in three models. Human milk oligosaccharides inhibited Campylobacter colonization in mice in vivo and inhibited invasive, pathogenic Campylobacter from binding to human intestinal mucosa ex viva. Inhibition by 2-linked glycans was also assessed in transgenic animals. Female mice had been engineered to carry a human α(1,2)-fucosyltransferase gene (FUT1) expressed under the control of a whey acidic protein promoter that induces the expression of 2-linked fucose antigens in mammary gland during lactation, and thus, in milk. As a control, non-transgenic parental mice were used. Their suckling pups were challenged with inocula of C. jejuni. Up to 90% of non-transgenic controls remained colonized during follow-up. Colonization of transgenic mice was transient, and the time of colonization was directly related to the inoculum (Table 2). Thus α(1,2)-linked fucosylated glycoconjugates in milk protected against Campylobacter infection in viva, confirming that the main intestinal ligands for Campylobacter are the H-2 histo-blood group antigens, and that milk fucosyloligosaccharides, specifically those containing α(1,2)-linked fucose, inhibit this binding.

TABLE 2
Campylobacter colonization in transgenic mice
carrying the FUT1 gene with the WAP promoter

| | Percentage of pups colonized by Campylobacter | | | |
|---|---|---|---|---|
| | Transgenic (FUT1) | | | |
| | Campylobacter inoculum | | | Non |
| Days after | (CFU/mL) | | | transgenic |
| infection | 510⁴ | 510⁸ | 510⁹ | Control 510⁸ |
| 1 | 40 | 30 | 90 | 100 |
| 3 | 40 | 60 | 50 | 90 |
| 5 | 10 | 40 | 20 | 70 |
| 7 | 0 | 0 | 0 | 70 |
| 9 | 0 | 10 | 10 | 67 |
| 11 | 0 | 0 | 0 | 70 |
| 13 | 0 | 0 | 0 | 70 |
| 15 | 0 | 0 | 0 | 90 |

Pups fed from transgenic mice cleared colonization 5 to 9 d after challenge with Campylobacter. Control pups from non-transgenic mice are unable to clear Campylobacter colonization.

The human milk fucosyloligosaccharide fraction has greater inhibitory activity on a weight basis against Campylobacter than its major component, 2′FL. The first step in searching for 2′FL homologs with greater inhibitory activity was to compare the activity of 2′FL, which contains a glucose on the reducing end, with 2′-fucosyl-N-acetyllactosamine (2′FLNAc), which is identical except that the sugar on the reducing end being N-acetylglucosamine. 2′FLNAc is the structural epitope found at the terminus of all higher homologs of 2′FL found naturally in human milk, including the macromolecular glycans. The 2′FLNAc is 18% more effective at inhibiting Campylobacter binding than 2′FL, consistent with the strongest inhibitors in human milk being the H-2 epitope expressed in macromolecules. Human milk contains a fucosylated macromolecular glycan that bound strongly to Campylobacter.

### Glycan Inhibition of Caliciviruses

The rabbit hemorrhagic disease virus (RHDV) specifically attaches to rabbit epithelial cells of the upper gastrointestinal and respiratory tracts through the H-2 epitope (Ruvoën-Clouet N. et al., 2000 J Virol, 74:11950-11954). VLPs of noroviruses (NVs) also bind to human gastro-duodenal epithelial cells derived from individuals of secretor phenotype, but not from non-secretor phenotype; non-secretors lack a functional α(1,2)-fucosyltransferase encoded by the FUT2 gene, indicating that the α(1,2)-fucose epitope is essential for binding. The specificity of NV binding was determined by specific blocking of the binding by human milk from a secretor, by monoclonal antibodies specific for H-1 and H-3 antigens, by synthetic oligosaccharide conjugates containing secretor antigens, and by treatment of the tissues with α(1,2)-fucosidase. Transfection of CHO cells with α(1,2)-fucosyltransferase cDNA allowed attachment of NV VLPs (Marionneau S. et al., 2002 Glycobiology, 12:851-856).

To determine the binding specificity of other strains of caliciviruses (CVs), saliva samples from 51 volunteers were tested for their ability to bind to eight recombinant capsid antigens representing seven genetic clusters of CVs (Huang 2003). The histo-blood group phenotypes were determined by monoclonal antibodies for Lewis and ABO antigens. Four patterns of binding were found: three of these (strains 387, NV, and MOH) bind secretors and one (strain 207) reacts with both non-secretors and secretors but prefers non-secretors. The three secretor-binding strains were further characterized based on the ABO antigen specificity: 387 recognizes all secretors (A, B, and O), NV recognizes A and O, and MOH recognizes A and B.

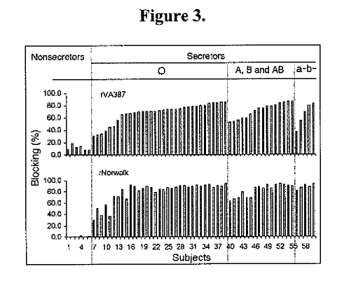

In 77 NV-challenged volunteers, 75% of the saliva samples from secretors, but none from the non-secretors, bound NV capsids (P<0.001). Secretors were almost 40 times more likely to become infected with NV than non-secretors, strongly suggesting that susceptibility to NV infection depends on secretor status. The ability of human milk to block binding of the most dominant strain of CV (VA387, genogroup II) to its receptor was tested (FIG. 3). The milks from 54 of the 60 mothers blocked this common human enteropathogen; only the milks from the six non-secretors failed to block binding, indicating that the binding was inhibited by α(1,2)-linked fucose epitopes. A preparation of the free human milk oligosaccharides failed to inhibit binding, but the human milk high-molecular-weight glycan did bind to Norwalk virus, and did block binding of Norwalk virus to its receptor. The data indicate that human milk contains fucosylated glycans that specifically block CV binding to histo-blood group antigen receptors.

### Example 3

### Fucosyl Glycans in Human Milk Inhibit Diarrhea

Maternal Lewis histo-blood group type is associated with heterogeneity in expression of fucosylation patterns of oligosaccharides and fucosylglycans in milk. This innate variation in milk oligosaccharide expression was used to examine the effectiveness of naturally occurring human milk glycans to protect nursing infants against diarrhea.

Milk samples were obtained weeks 2-5 postpartum from 93 Mexican mothers; the mothers and infants were followed from birth up to 2 yr postpartum. The clinical histories of the infants were recorded prospectively, and concentrations of individual oligosaccharides in these milk samples were measured. The most common oligosaccharides in maternal milk were those that were fucosylated, comprising 73% (50%-92%) of total oligosaccharide. Of these, 2′FL, the oligosaccharide homolog of the Lewis H-2 epitope, was the most common in the milks of all mothers. The mean concentration of 2′FL in milk was 3854±108 nmol/mL (34% of total fucosylated oligosaccharides), but varied greatly among mothers according to their Lewis blood group type.

Breastfed infants who were infected with STEC (i.e., exhibited stable toxin positive E. coli in their stools) were divided into two categories: Those exhibiting clinical symptoms of diarrhea (symptomatic), and those not exhibiting symptoms (asymptomatic). Those children who had symptoms of diarrhea were consuming milks with low relative amount of 2-linked oligosaccharides: the ratio of α(1,2)- to α(1,3/4)-linked fucosylated oligosaccharides in their mothers' milk was 3.9±0.7 [SE], (n=4), significantly lower than that of milks being consumed by infants who were also infected with STEC, but did not develop diarrhea (7.6±1.0, n=43), or uninfected controls (7.5±1.0, n=46) (P<0.01). Thus, higher concentrations of α(1,2)-linked oligosaccharides in milk (reflecting higher consumption by the infant) protects the infant against ST-associated diarrhea. This observation illustrates that variable expression of fucose epitopes in human milk is associated with differences in the ability of the milk to protect infants against a pathogen.

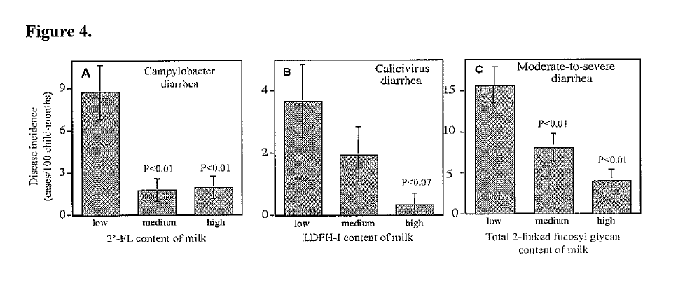

In these 93 nursing children consumption of milk containing high levels of 2′FL, but not of other oligosaccharides, was significantly associated with protection against Campylobacter diarrhea (Poisson regression, P=0.004; FIG. 4 panel A). Consumption of milk containing high amounts of LDFH-I (another 2-linked fucosyloligosaccharide, which is the oligosaccharide homolog of the Le^{b} epitope), but not of the other oligosaccharides, was associated with protection against calicivirus diarrhea (Poisson regression, P=0.012; FIG. 4 panel B). More generally, infants consuming milk relatively high in all measured 2-linked oligosaccharides were significantly (P<0.001) protected against diarrhea of all causes FIG. 4 panel C). Thus, human milk containing high levels of specific oligosaccharides are associated with decreased risk of pathogen-specific diarrhea and diarrhea overall. For some pathogens, these milk oligosaccharides per se, as well as high-molecular-weight milk components containing these specific glycan epitopes, inhibit the pathogens in vivo and in vitro; in others, only the high-molecular-weight components in milk that contain the relevant epitope exhibit inhibition. Because both the oligosaccharide and the high-molecular-weight glycans that share the same glycan epitope are synthesized by the same fucosyltransferase, the relationship between the oligosaccharide expression in milk and decreased risk in the infant reflects the synthetic capacity of the mother for both the free oligosaccharide and the homologous glycan. Specific human milk oligosaccharides protect breastfed children from disease by inhibiting binding of pathogens to the child's intestinal receptors.

### Example 4

### GDP-Fucose Synthesis in the K. lactis Cytoplasm

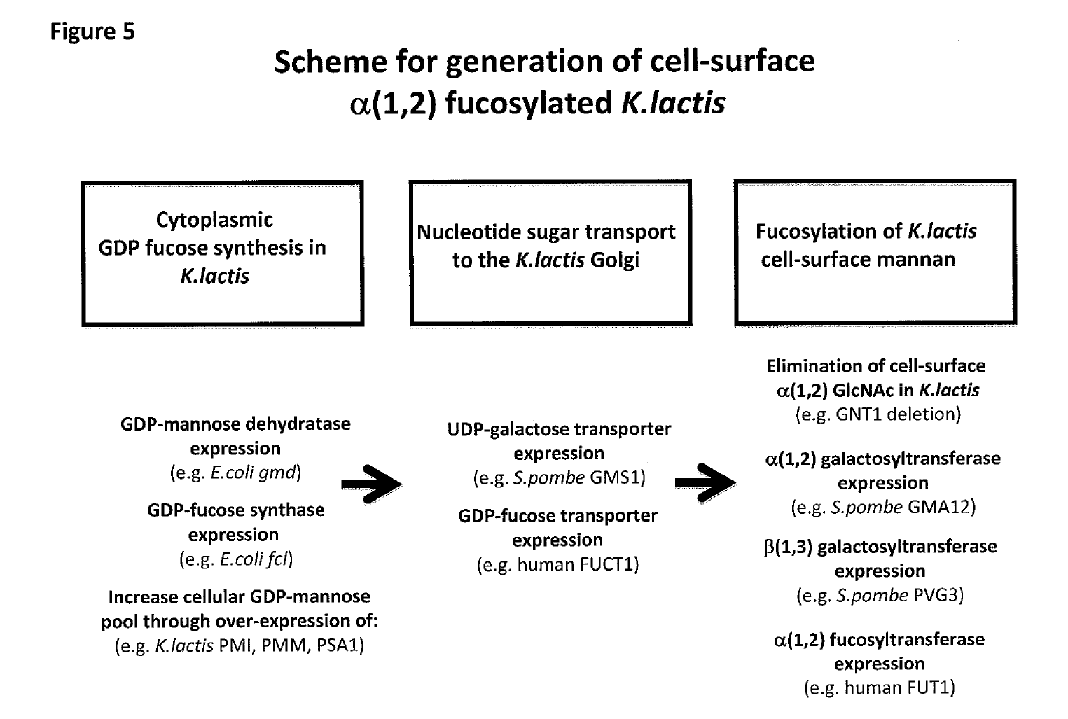

The yeast Kluyveromyces lactis, already a common constituent of human foods such as cheeses, is known to survive well in the conditions found in the human alimentary tract. Kluyveromyces lactis cells, modified through the steps outlined below (and summarized in FIG. 5) to display α(1,2) fucosylated glycans on their cell surface, when taken orally either as specific preparations, or as constituents of foods and beverages, act as high avidity decoys within the gut, adsorbing pathogens and providing resistance to infection. Kluyveromyces lactis is modified as follows to provide it with a new ability to display α(1,2) fucosylated glycans on its cell surface.

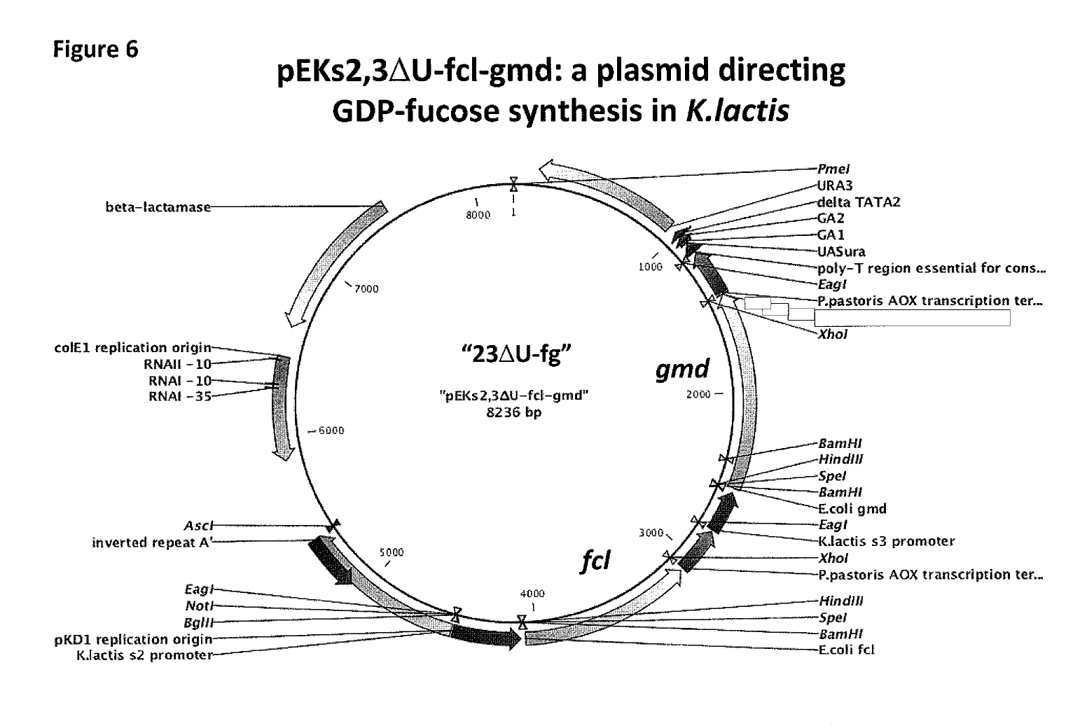

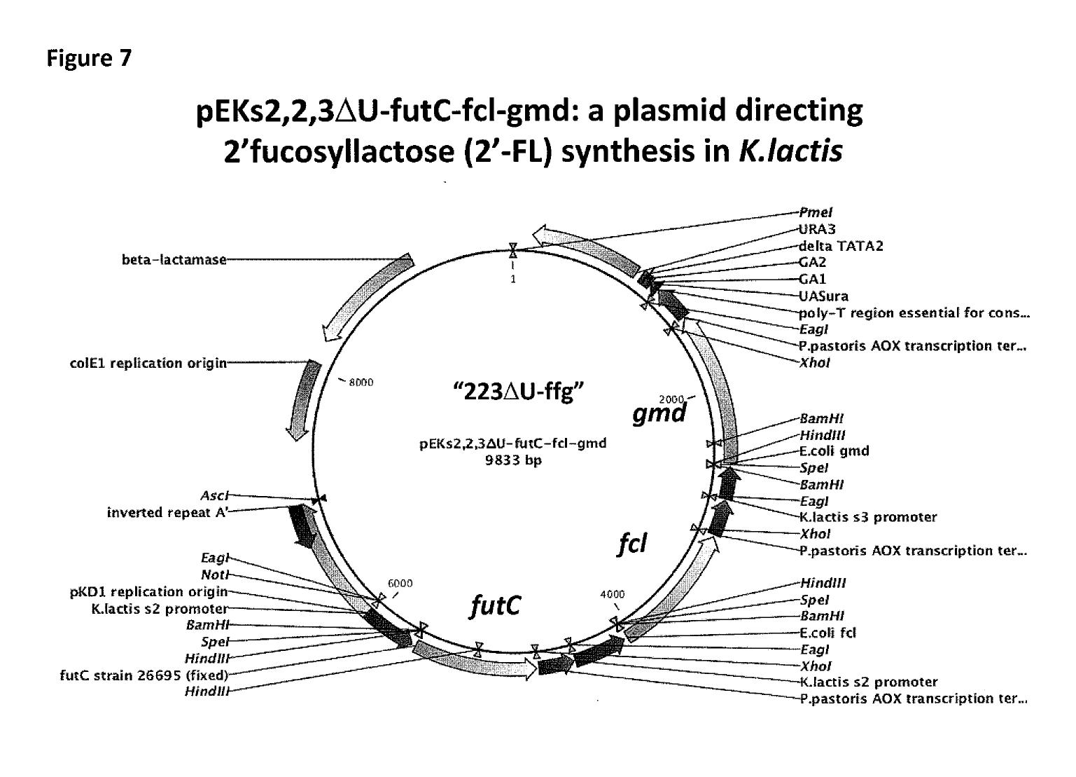

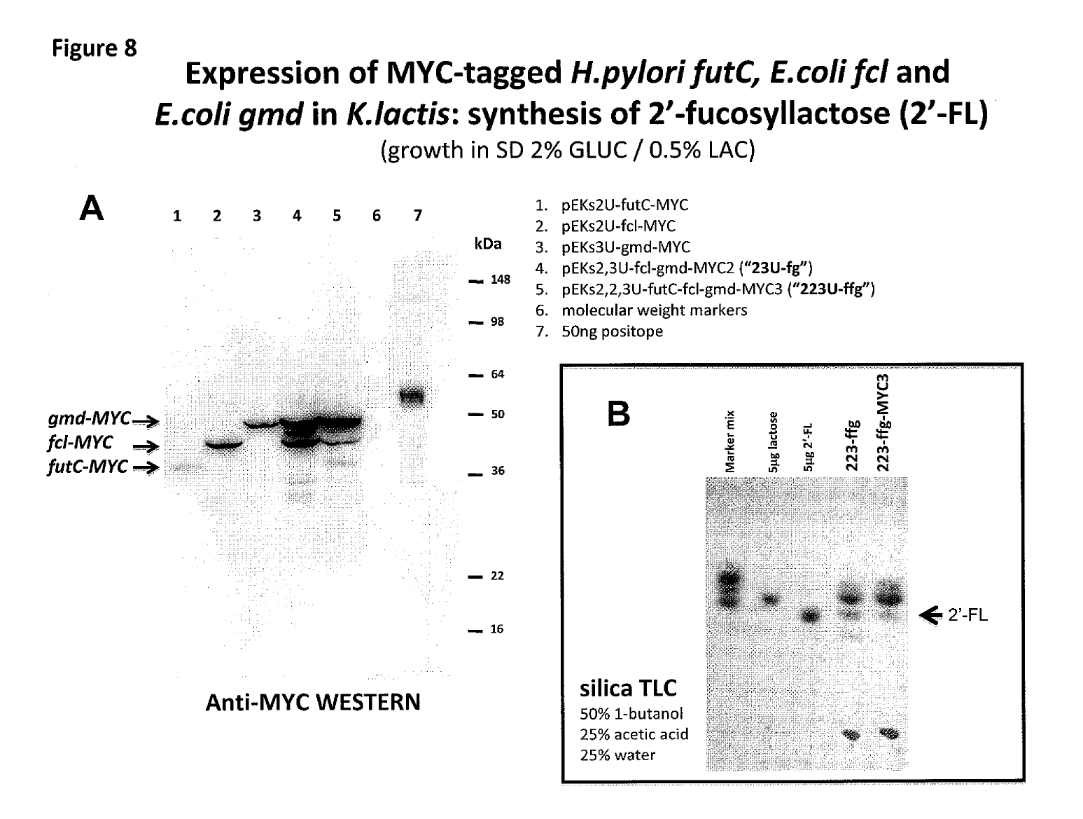

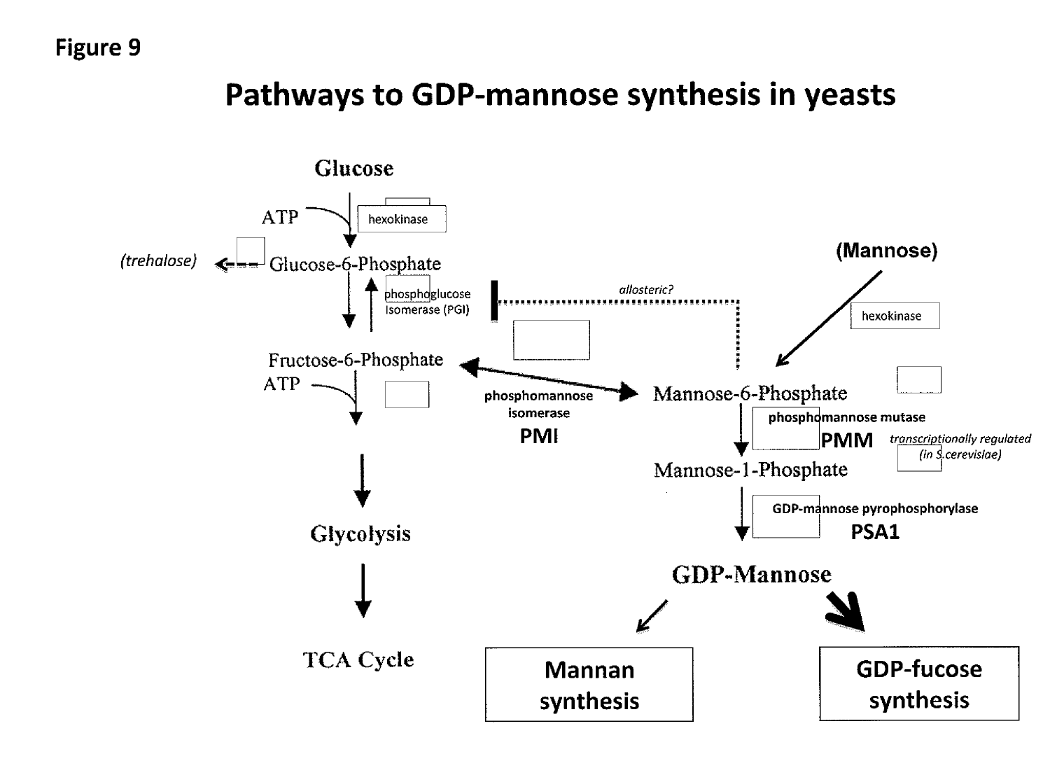

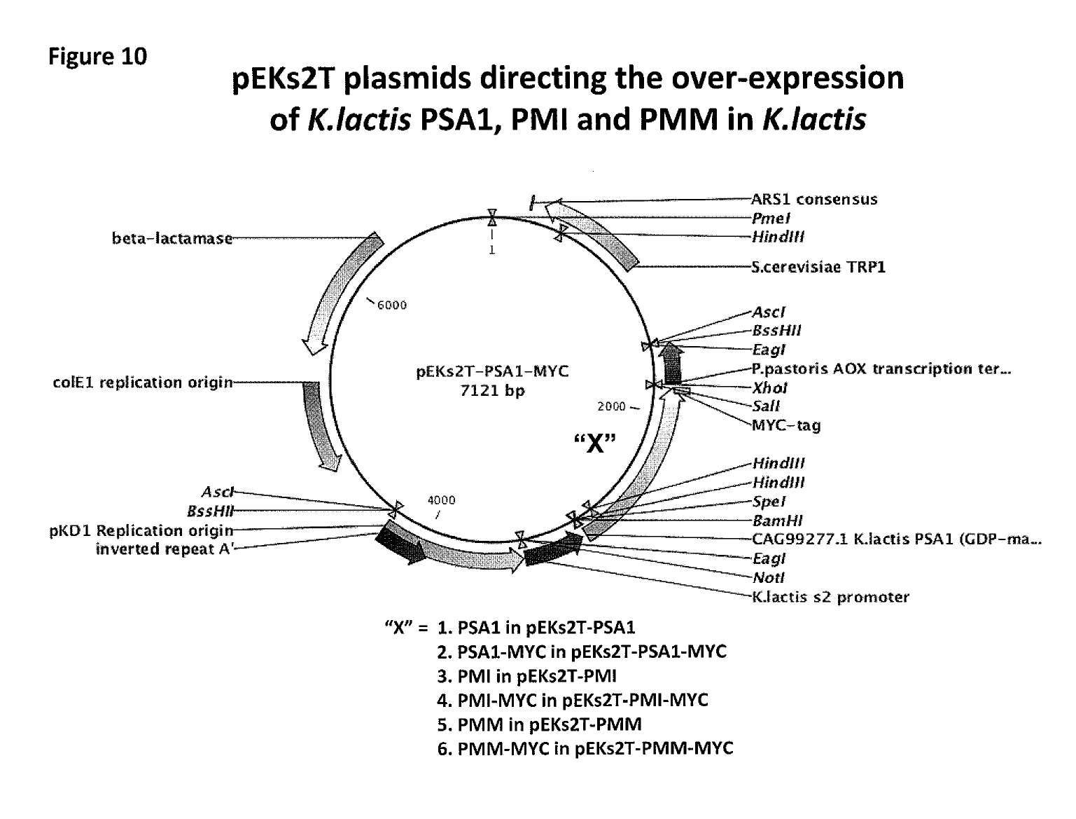

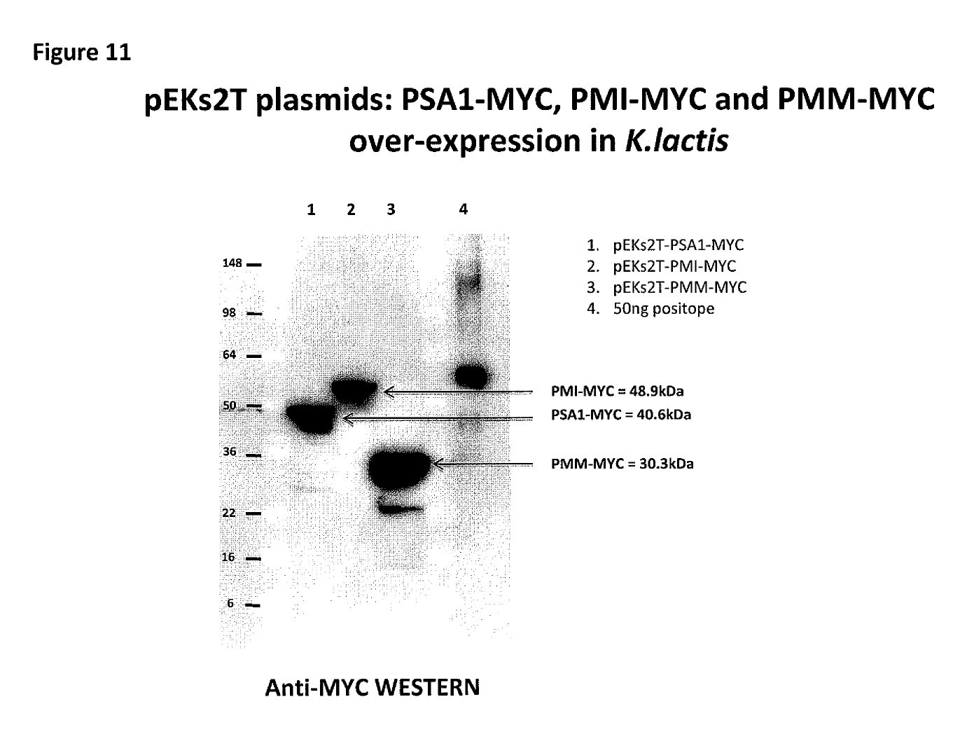

Kluyveromyces lactis lacks the ability to make GDP-fucose, a key precursor for synthesis of fucosylated glycans. Thus, an essential first step is to provide K. lactis with the means to generate this nucleotide-sugar. The K. lactis cell wall comprises glycoproteins (mannoproteins) that carry covalently linked mannose and glucose/glucosamine polymers (Gemmill T. R. and Trimble R. B., 1999 Biochim Biophys Acta, 1426:227-237). In general, yeast mannan synthesis requires an abundant supply of precursors; in particular a large amount of GDP-mannose is produced in the yeast cell cytoplasm for transport into the Golgi where it is used in mannosylation reactions. Mutations eliminating or materially reducing GDP-mannose synthesis or mannosylation in yeasts are lethal or deleterious. In the surface-engineered strains of the current invention a portion of cellular GDP-mannose flux is diverted toward the production of cytoplasmic GDP-fucose, while overall cell viability is maintained. GDP-mannose is converted to GDP-fucose in a three-step reaction catalyzed by two enzymes; GDP-D-mannose dehydratase (GMD) and GDP-L-fucose synthetase (GFS). Genes encoding these enzymes have been isolated from a number of organisms, including E. coli, Helicobacter pylori (Wu B. et al., 2001 Biochem Biophys Res Commun, 285:364-371) and humans (Sullivan F. X. et al., 1998 J Biol Chem, 273:8193-8202). These genes were introduced into K. lactis (strain “K25”: MATa, uraA trp1 lysA1 lac4-8 [pKD1+]) either positioned on plasmids, such as the plasmid pEKs2,3ΔU-fcl-gmd (FIG. 6, SEQ ID:1), or as stable chromosomal insertions. pEKs2,3ΔU-fcl-gmd is a shuttle vector capable of stable replication in E. coli (by virtue of a pUC-18 origin of replication and an ampicillin resistance selectable marker gene), or alternatively in K. lactis (by virtue of a pKD-1 origin of replication, allowing replication in pKD-1⁺ strains, and a S. cerevisiae URA3 selectable marker gene). pEKs2,3ΔU-fcl-gmd carries two strong K. lactis promoters, s2 and s3, which constitutively drive the expression of E. coli fcl and gmd genes, respectively. Downstream of both fcl and gmd in pEKs2,3ΔU-fcl-gmd are positioned copies of a transcription terminator based on that of the alcohol oxidase gene of P. pastoris (AOXter). A linear ˜4.5 kb DNA fragment carrying s2-fcl, s3-gmd, and S. cerevisiae URA3 can be conveniently prepared from pEKs2,3ΔU-fcl-gmd by digestion with NotI and PmeI. In some embodiments of the current invention, this fragment was transformed into K. lactis by electroporation and efficiently incorporated into genomic DNA through non-homologous recombination, a mechanism that is extremely active in this organism. E. coli GMD and GFS enzymes have previously been introduced into the yeast Saccharomyces cerevisiae and shown to direct the synthesis of GDP-fucose (Mattila P. et al., 2000 Glycobiology, 10:1041-1047). Several assays to determine GDP-fucose levels are known in the art. To demonstrate GDP-fucose production in engineered K. lactis, we chose to combine enzymatically GDP-fucose was combined with lactose in the K. lactis cytoplasm to generate 2′-fucosyllactose (2′-FL), the presence of which was conveniently assayed by thin layer chromatography (TLC). FIG. 7 shows pEKs2,2,3ΔU-futC-fcl-gmd, a plasmid based on pEKs2,3ΔU-fcl-gmd, but with an additional s2-futC-AOXter expression cassette inserted 5′- of fcl. (futC is an α(1,2) fucosyl transferase gene from Helicobacter pylori). FIG. 8, panel A, shows individual expression of MYC-tagged futC, fcl and gmd (lanes 1-3) directed by plasmids based on “pEKs2,3ΔU-fcl-gmd” but that carry only single gene cassettes. Co-expression in the same cell of fcl and gmd, directed by pEKs2,3ΔU-fcl-gmd, is shown in lane 4. Co-expression of fcl, gmd and futC in the same cell, directed by pEKs2,2,3ΔU-futC-fcl-gmd, is shown in lane 5. FIG. 8, panel B, shows the production of 2′-FL in cells co-expressing fcl, gmd and futC and grown in the presence of lactose. The production of 2′-FL demonstrates that GDP-fucose was successfully made from GDP-mannose in K. lactis. To mitigate the impact on cell viability due to depletion of cellular GDP-mannose, the cellular GDP-mannose pool was enhanced in K. lactis through over-expression of the K. lactis genes encoding three essential enzymes in the flow of carbon to GDP-mannose from central glucose metabolism: phosphomannose isomerase (PMI, KLLA0D13728g), phosphomannose mutase (PMM, SEC53) and GDP-mannose pyrophosphorylase (PSA1, (Uccelletti D. et al., 2005 FEMS Yeast Res, 5:735-746)). FIG. 9 shows the pathway of GDP-mannose synthesis in yeasts, and the positioning of these three enzymes in this scheme. External mannose in the growth medium can also directly enter into this pathway, as shown in FIG. 9, and mannose inclusion in growth media is an option in some embodiments. FIG. 10 shows pEKs2T-PSA1-MYC, a plasmid based on pEKs2,3ΔU-fcl-gmd, but with a single s2-PSA1-MYC cassette, and with the S. cerevisiae URA3 gene replaced by S. cerevisiae TRP1. In K. lactis strain “K25” TRP1- and URA3-carrying plasmids can replicate and co-exist in the same cell in dual selective media. pEKs2T-based vectors were constructed for MYC-tagged and untagged versions of PMI, PMM and PSA. A single pEKs2T-based vector was also constructed that made all three enzymes in the same cell simultaneously, and at the same time as fcl and gmd were expressed by pEKs2,3ΔU-fcl-gmd. FIG. 11 shows robust over-expression of MYC-tagged PMI, PMM and PSA1 utilizing pEKs2T-based plasmid constructs.

The cytoplasmic pool of GDP-mannose may be elevated in certain non-lethal mutants of the yeast GDP-mannose Golgi transporter, VRG4 (Gao X. D., 2001 J Biol Chem, 276:4424-4432). Use of these mutations also allows for increased production of GDP-fucose GMD/GFS (gmd/fcl) are sensitive to feedback inhibition, which may limit the overall yield of cytoplasmic GDP-fucose achieved. However, provision of a GDP-fucose “sink” relieves this inhibition (e.g., through providing an enzyme in the cell cytoplasm to consume GDP-fucose in generating a fucosylated product, or a transporter to move the GDP-fucose into a different cellular compartment (see below)).

Long-term stability of engineered strains is important in commercial manufacturing, so consideration is given to chromosomal versus episomal location of the introduced recombinant genes, and to the choices of selectable markers for episomal maintenance.

Species origin for the GMD and GFS introduced into K lactic is selected by GRAS considerations, i.e., genes derived from non-pathogens is preferred.

### Example 5

### Transport of Cytoplasmic GDP-Fucose and UDP-Galactose into the K. LACTIS Golgi

In eukaryotes, glycosylation of secreted proteins occurs in the ER and Golgi, where membrane-bound glycosyltransferases utilize luminal GDP-, UDP- or CMP-sugar substrates (nucleotide-sugars) to glycosylate suitable protein acceptors. The various nucleotide-sugars are exported to the lumen of the ER and Golgi from the cell cytoplasm by transmembrane nucleotide-sugar transporter proteins (NSTs). NSTs can be monospecific, transporting only one particular nucleotide-sugar, or di- or multi-specific, exhibiting relaxed substrate specificity. Particular organisms may entirely lack the ability to transport particular nucleotide-sugars, depending on the types of glycan they have evolved to elaborate.

All human H— (and Lewis) antigens contain both a fucose and a galactose moiety. Steps to engineer cytoplasmic synthesis of GDP-fucose in K. lactis were described in example 4, and cytoplasmic UDP-galactose is a normal K. lactis metabolite. However, K. lactis has not been shown to possess intrinsic abilities to import either GDP-fucose or UDP-galactose into the Golgi. The invention provides for the development of a strain of K. lactis that is competent for the Golgi import of both GDP-fucose and UDP-galactose.

UDP-galactose transport to the golgi has not been described for wild type K. lactis. The complete genome sequence was found to include the presence of a putative gene (locus Q6CR04, Homolog of UDP-galactose Transporter (HUT1)) that possesses significant homology to other characterized UDP-galactose transporters. Moreover, Saccharomyces cerevisiae contains a homologous gene (HUT1), 53% identical at the amino acid level to K. lactis HUT1, which has been experimentally linked to an increased ability to incorporate galactose into glycans (in the presence of a heterologous galactosyltransferase) (Kainuma M. et al., 2001 Yeast, 18:533-541). The innate ability of K. lactis to transport UDP-galactose to the Golgi can be readily checked utilizing radiolabelled UDP-galactose and K. lactis golgi membrane vesicles prepared using published procedures (Gao, X D, Nishikawa, A and Dean, N, J Biol Chem 276:6, 4424-32 (2001)). Additional UDP-galactose transporter activity is provided to K. lactis by over-expression of a heterologous UDP-galactose transporter gene using an expression cassette, vector and strain as outlined in example 4. Examples of heterologous UDP-galactose transporters are used are the human UDP-galactose transporter (UGT1) (Miura N. I et al., 1996 J Biochem, 120:236-241), the S. pombe UDP-galactose transporter (GMS1) (Kainuma, M, Ishida, N, Yoko-o, T, Yoshioka, S, Takeuchi, M, Kawakita, M and Jigami, Y, Glycobiology 9:2, 133-41 (1999)), or the S. cerevisia UDP-galactose transporter (HUT1) described above.

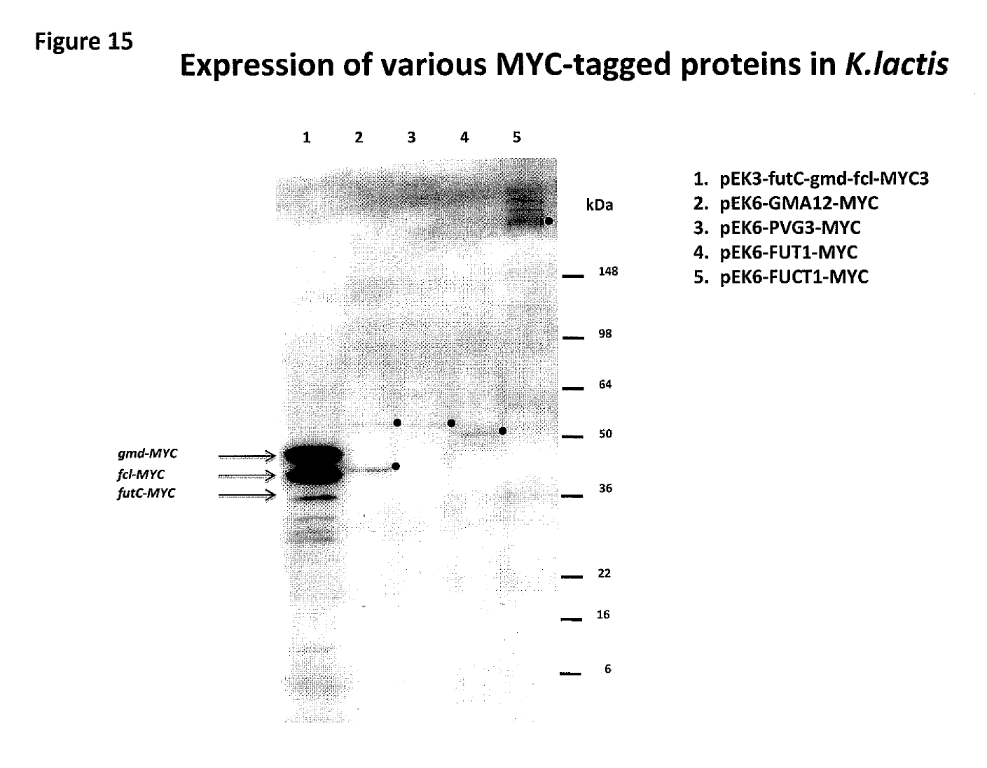

Golgi import of GDP-fucose in humans is achieved through a fucose-specific NST, FUCT1, mutations in which are responsible for leukocyte adhesion deficiency II. The direct approach to ensure Golgi transport of GDP-fucose in K. lactis is to express human FUCT1 in the organism. FIG. 15, lane 5 demonstrates expression of MYC-tagged human FUCT1 in K. lactis, utilizing using an expression cassette, vector and strain as outlined in example 4. A broad range of known GDP-fucose transporters are optionally expressed in K. lactis in order to transport GDP-fucose into the golgi, (e.g., GDP-fucose transporters from other mammalian species such as dogs, mice, pigs and cows, or from birds (e.g. chickens) or fish (e.g. zebrafish), or many other species. An alternative approach is the introduction into K. lactis of a multi-specific NST, such as the Leishmania LPG2 protein (Segawa H. et al., 2005 J Biol Chem, 280:2028-2035). Since LPG2 has the ability to transport both GDP-mannose and GDP-fucose, it is a useful tool in balancing the relative synthesis and transport of these two nucleotide sugars. Controlling this balance is important for maintaining strain viability while maximizing overall fucosylated product yield. One immediate indicator of success for Golgi GDP-fucose transport in the engineered strain is increased total cellular GDP-fucose levels through relief of product feedback inhibition on cytoplasmic GDP-L-fucose synthetase. A more direct indication of functional activity of human FUCT1, or Leishmania LPG2, or human UGT1 in K. lactis is measurements of labeled nucleotide-sugar uptake into purified yeast Golgi vesicle preparations (Gao, X D, Nishikawa, A and Dean, N, J Biol Chem 276:6, 4424-32 (2001)).

### Example 6

### Elimination of α(1,2) GlcNAc in K. lactis Mannans

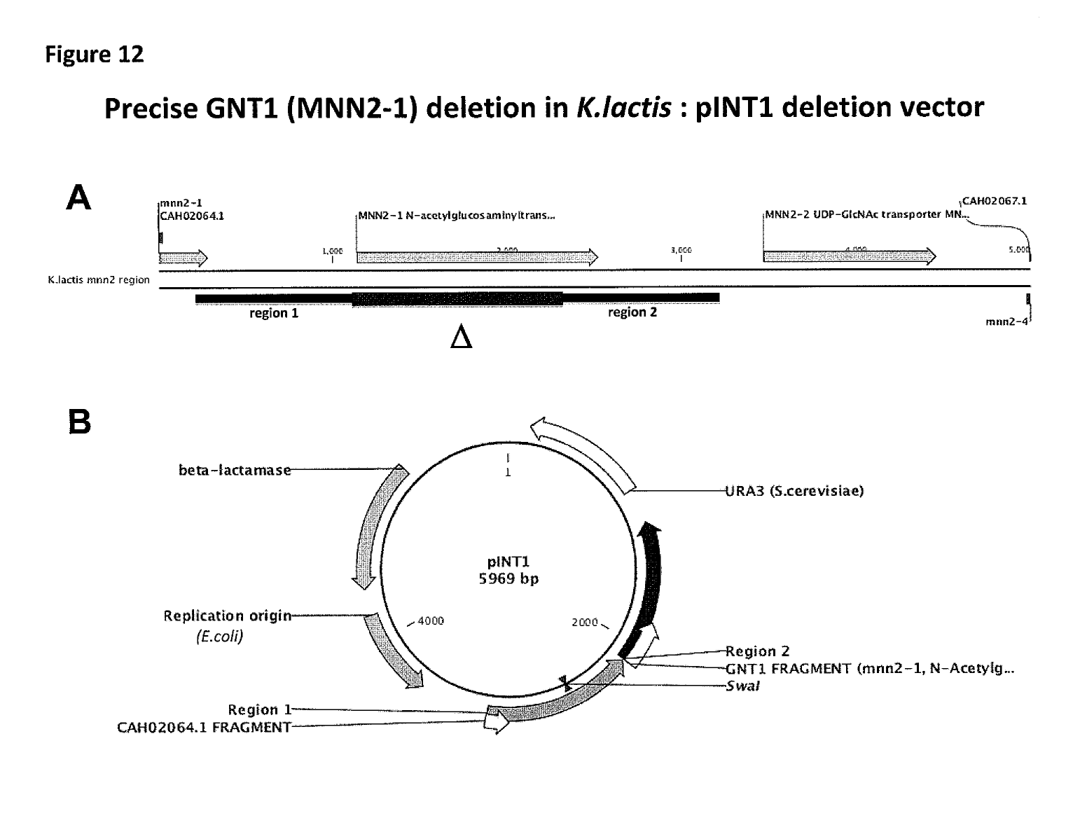

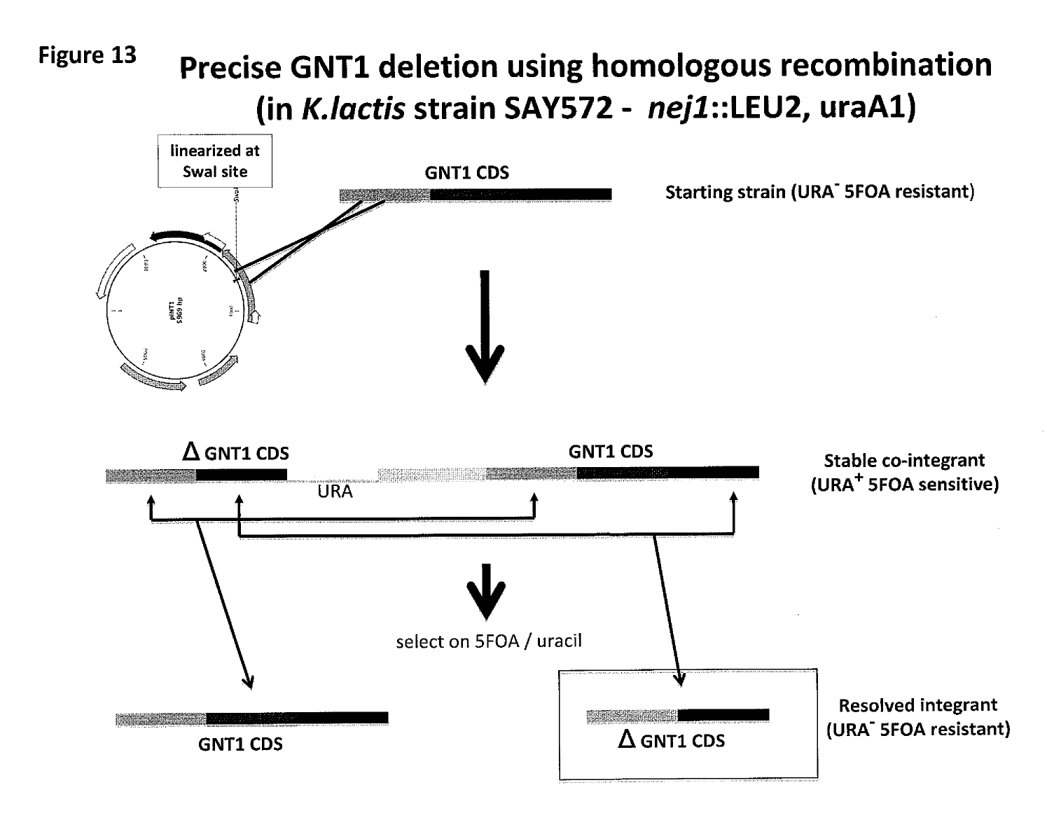

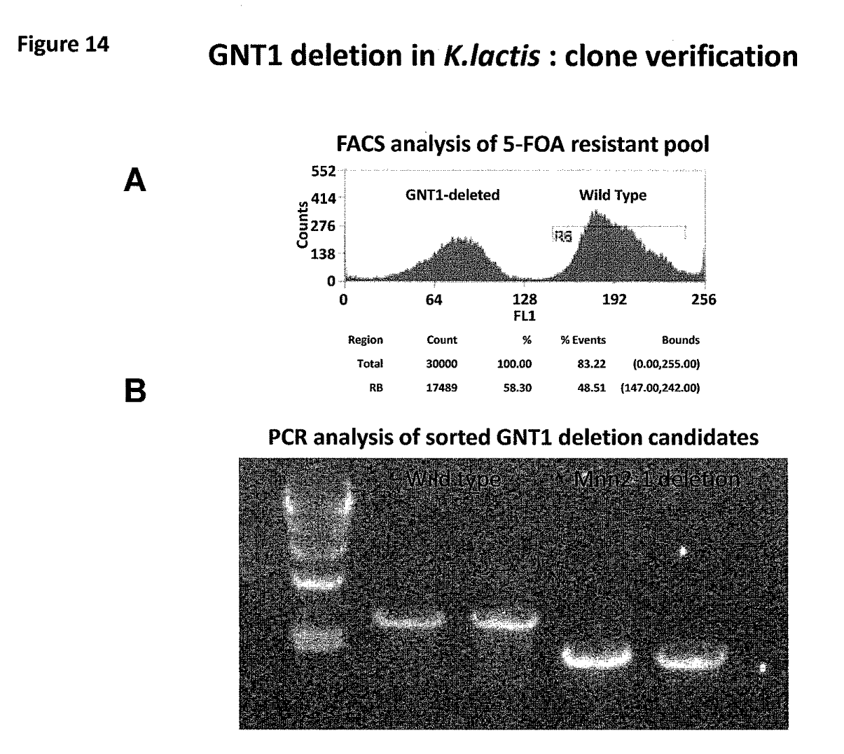

The mannans of the K. lactis cell wall comprise linear α(1,6)-linked mannose backbones to which are attached branched α(1,2)-linked mannose side chains. Many of these branches are terminated by a single α(1,3)-mannose, with the penultimate α(1,2) mannose group at termini frequently being modified with α(1,2)-linked N-acetylglucosamine (Guillen E. et al., 1999 J Biol Chem, 274:6641-6646). This α(1,2) GlcNAc is not a suitable acceptor for galactose addition, and thus it is desirable to eliminate GlcNAc from K. lactis mannan to clear the way for galactose and fucose additions. The formation of terminal α(1,2) GlcNAc is prevented by the introduction of either one of two previously described K. lactis mutations, mnn2-1 and mnn2-2 (Smith W. L. et al., 1975 J Biol Chem, 250:3426-3435). The former abolishes N-acetylglucosaminyltransferase activity, the latter removes UDP-GlcNAc Golgi transport activity. K. lactis strains carrying either mutation are viable, while the presence of either results in mannan lacking terminal α(1,2)-GlcNAc. FIG. 12, panel A illustrates the K. lactis genome surrounding the GNT1 (MNN2-1) locus, encoding N-acetylglucosaminyltransferase. Approximately 3 kb of K. lactis chromosomal DNA, including the entire GNT1 (MNN2-1) gene and ˜1 kb of 5′ and 3′ flanking sequences, was cloned into an E. coli plasmid. A precise 1000 bp DNA segment designated “A” in the figure, whose 5′-end was the GNT1 methionine initiator codon, was then deleted to generate the integration vector pINT-1 (FIG. 12, panel B). This vector, which is was not capable of replication in K. lactis, was linearized with restriction endonuclease SwaI as indicated, and transformed by electroporation into K. lactis strain SAY572 (uraA1 trp1 leu2 lysA1 metA1 nej::LEU2) (Kegel, A, Martinez, P, Carter, S D and Aström, SU. Nucleic Acids Res 34:5, 1633-45 (2006)). SAY572 is deficient in non-homologous recombination, and URA⁺ colonies derived from the transformation survived by incorporating the linear pINT-1 by homologous recombination at the GNT1 locus, generating a cointegrant structure as outlined in FIG. 13. Following an outgrowth on non-selective medium, resolved, URA⁺ cointegrants were isolated by plating onto medium containing 5FOA and uracil. FIG. 14, panel A, shows a FACS scan of a pool of recovered URA⁻ clones (stained with fluorescently labeled Griffonia simplicifonia lectin II, GSII, specific for GlcNAc). The two observed peaks were as expected, one (FACS bright) represented clones that had resolved back to wild type, the second peak (FACS dark) were clones that had resolved to maintain the 1000 bp GNT1 deletion. FIG. 14, panel B shows confirmation of this deletion by PCR in two clones purified from the FACS dark population. These clones do not incorporate GlcNAc into their mannan.

### Example 7

### Addition of α(1,2) Galactose Residues onto K. lactis Surface Polymannose

A galactose molecule is added to the K. lactis surface polymannose (in the GNT1 null mutant background of example 6) to serve as a foundation for subsequent galactose and fucose additions. Schizosaccharomyces pombe possesses an α(1,2) galactosyltransferase, the product of the gma12 gene that is able to link galactose to mannose acceptors (Chappell T. G., et al., 1994 Mol Biol Cell, 5:519-528). GMA12 enzyme incorporates galactose into cell wall mannans in S. cerevisiae (Kainuma M. et al., 1999 Glycobiology, 9:133-141). FIG. 15, lane 2, demonstrates expression of MYC-tagged S. pombe GMA12 in K. lactis, utilizing using an expression cassette, vector and strain as outlined in example 4. Galactose incorporation by GMA12 is more efficient in a mnn1 strain background (i.e., deficient in a (1,3) mannosyltransferase) (Kainuma M. et al., 1999 Glycobiology, 9:133-141). K. lactis mnn1 mutants are employed here to optimize the yield of α(1,2) galactose incorporated into polymannose.

### Example 8

### Addition of Beta (1,3) Galactose to Surface α(1,2) Galactose Residues

Next, β(1,3) galactose is added to the α(1,2) galactose previously incorporated in Example 7 (above). Schizosaccharomyces pombe possesses an enzyme well suited to this purpose, a β(1,3) galactosyltransferase that is the product of the PVG3 gene (Andreisheheva E. N. et al., 2004 J Biol Chem, 279:35644-35655). This gene is introduced into the engineered K. lactis strain from Example 7. FIG. 15, lane 3, demonstrates expression of MYC-tagged S. pombe PVG3 in K. lactis, utilizing using an expression cassette, vector and strain as outlined in Example 4. Successful incorporation of galactose into K. lactis mannan is monitored using lectin-binding assays. (e.g., Griffonia simplificolia Lectin I-B₄ binding for α-linked galactose, Peanut agglutinin binding for beta-linked galactose).

### Example 9

### Expression of Human H Epitopes on the K. lactis Cell Surface and Optimization of H-Epitope Levels

H-antigen is expressed on K. lactis cell surface by the addition of α(1,2) fucosyl-groups to the β(1,3) galactose-modified mannan of the strain generated in Example 8. α(1,2) fucosylation is accomplished in humans by either one of two type II transmembrane Golgi α(1,2) fucosyltransferases that are the products of two distinct but related genes, Homo sapiens fucosyltransferase 1 (galactoside 2-alpha-L-fucosyltransferase; FUT1) and Homo sapiens fucosyltransferase 2 (FUT2) (Sarnesto A. et al., 1992 J Biol Chem, 267:2737-2744; Larsen R. D. et al., 1990 Proc Natl Acad Sci USA, 87, 6674-6678; Kelly R. J. et al., 1995 J Biol Chem, 270:4640-4649). FUT1 (also known as the H-gene) is expressed in human mesenchymal tissues; FUT2 (also known as the secretor gene or Se) is expressed in human epithelial tissues. FUT2 is responsible for α(1,2) fucosylation of glycans in secretions (tears, saliva, milk etc) and on epithelial/mucosal surfaces, and is the enzyme responsible for the fucosylation that has been shown to protect against pathogen adherence in humans. FUT1 and FUT2 are only 68% identical at the amino acid level across their catalytic domains; however they have been directly demonstrated to be quite similar enzymatically in terms of substrate preference, despite earlier studies that had predicted that substantial differences between them might exist. Each enzyme is tested individually in K. lactis, with H-antigen expression being monitored by Ulex europaeus lectin binding or by ELISA. FUT1 and FUT2 possess minor difference substrate specificity. Each or both gene products may be expressed and the level or ratio of expression manipulated to maximize final yields of H-antigen. FIG. 15, lane 4, demonstrates expression of MYC-tagged human FUT1 in K. lactis, utilizing using an expression cassette, vector and strain as outlined in Example 4.

Several additional approaches are optionally utilized to yield optimization.
use of mnn1 and mnn2 mutants (see examples 6 and 7 above)
use of VRG4 mutants (see example 4 above)
use of phosphomannose isomerase (PMI), phosphomannose mutase (PMM) and GDP-mannose pyrophosphorylase (PSA1) over-expression (see example 4 above)
tailoring of enzyme levels through control of expression parameters, e.g. the use of stringer or weaker promoters in the expression cassettes described in example 4 (above)
Cell yields are optimized under controlled conditions in a bioreactor by manipulating variables such as base growth medium, carbon source, inoculum preparation procedure, and various physical growth conditions (i.e., aeration/agitation rate, culture temperature, culture pH). Statistical methods for rapid optimization of multiple variables in fermentation processes are well established and are employed here (Kalil S. J. et al., 2001 Appl Biochem Biotechnol, 94:257-264).

### Example 10

### Biological Testing

The engineered strain of K. lactis is used as a probiotic product. The engineered strain carries on its cell surface polyvalent α(1,2) fucosylated glycan structures that bind efficiently to adherence determinants of a number of pathogenic organisms. The engineered strain when ingested acts as a decoy for gut pathogens. The ability of the surface α(1,2) fucosylated K. lactis strain generated in this invention prevents adherence and infection by pathogens. The K. lactis strain is tested for efficacy in suitable animal models of gut infection (Newell D. G., 2001 Symp Ser Soc Appl Microbiol, 57S-67S).

Direct Binding of Campylobacter to Engineered K. lactis Cells

The ability of Campylobacter to adhere to engineered K. lactis is assessed microscopically. Wild type and engineered K. lactis are incubated with bacteria, washed, and bound bacteria visualized and quantified under a phase contrast microscope.

### Solid-Phase Assays

The ability of Campylobacter to adhere to histo-blood group antigens, and the inhibition of this binding by various agents, is assessed in bacterial-binding Western blot assays using DIG-labeled bacteria. Mucin is run on lanes of SDS-PAGE gels and then electro-blotted onto nitrocellulose membranes. Membranes are then washed, immersed in a DIG-labeled bacterial suspension, and bound bacteria visualized with alkaline phosphatase-conjugated anti-DIG antibody stained with X-Phosphate and nitroblue tetrazolium. In a solid-phase Norwalk Virus binding inhibition assay, saliva obtained from secretors is purified and placed at the bottom of a 96-well plate. The binding of Norwalk Virus capsid is measured using known methods. The effectiveness of test agents in inhibiting adherence in both assays is assessed readily. In the case of the engineered K. lactis strain generated here, the effectiveness of a pre-incubation step with pathogen to reduce the observed signal in each assay is assessed.

### Mammalian Cell-Binding Assay

α(1,2) fucosylated glycans generated as described herein are tested for their ability to inhibit binding of Campylobacter to genetically modified CHO cells. Bacterial binding, in the presence or absence of a pre-incubation step with engineered K. lactis, to CHO cells transfected with human α(1,2) fucosyltransferase (FUT1) and expressing cell-surface α(1,2) fucosylated proteins, is compared to vector only-transfected and parental CHO cells. Data are interpreted as percent inhibition of bacteria association to cells relative to positive controls.

### In Vivo Testing

Campylobacter colonization in vivo is achieved in the inbred mouse strain, BALB/c (Blaser M. J. et al., 1983 Infect Immun, 39:908-916). Inhibition or reduction of infection by the surface α(1,2)-fucosylated K. lactis strain generated as described herein is assessed in two modes, i.e., by prophylaxis and by post-infection treatments. Engineered yeast, wild type yeast or vehicle are administered to mice p.o., either 2d prior or 7d subsequent to inoculation with 10⁸ CFU of the Campylobacter jejuni invasive strain 287ip. Levels of colonization are monitored by measuring Campylobacter CFU in stool samples.

What is claimed is:

1. A composition comprising a yeast cell, said yeast cell comprising an exogenous nucleic acid molecule encoding GDP-mannose-4,6-dehydratase (GMD) and an exogenous nucleic acid molecule encoding GDP-L-fucose synthetase (GFS).

2. The composition of claim 1, wherein said GMD is selected from the group consisting of H. pylori GMD, E. coli GMD, and Homo sapiens GMD.

3. The composition of claim 1, further comprising a nucleic acid molecule encoding GDP-mannose pyrophosphorylase.

4. The composition of claim 1, further comprising a nucleic acid molecule encoding phosphomannose isomerase.

5. The composition of claim 1, further comprising a nucleic acid molecule encoding phosphomannose mutase.

6. The composition of claim 1, wherein said yeast cell further harbors a nucleic acid molecule encoding a nucleotide sugar transporter (NST).

7. The composition of claim 6, wherein said NST is selected from the group consisting of human FUCT1, human UGT1, and S. cerevisiae HUT1.

8. The composition of claim 1, wherein said yeast cell over-expresses an endogenous NST.

9. The composition of claim 6, wherein said yeast cell lacks terminal N-acetylglucosaminyltransferase activity or UDP-N-acetylglucosamine transporter activity.

10. The composition of claim 1, wherein said yeast cell is a Kluyveromyces lactis yeast cell.

11. The composition of claim 9, wherein said yeast cell is a Kluyveromyces lactis yeast cell carrying a mnn2-1 or mnn2-2 mutation.

12. The composition of claim 9, wherein said yeast cell further harbors a nucleic acid molecule encoding an α(1,2) galactosyltransferase.

13. The composition of claim 12, wherein said nucleic acid molecule encoding an α(1,2) galactosyltransferase is Schizosaccharomyces pombe gma12.

14. The composition of claim 12, wherein said yeast cell further harbors a nucleic acid molecule encoding a β(1,3) galactosyltransferase.

15. The composition of claim 12, wherein said nucleic acid molecule encoding a β(1,3) galactosyltransferase is Schizosaccharomyces pombe PVG3.

16. The composition of claim 12, wherein said yeast cell further harbors a recombinant nucleic acid molecule encoding a fucosyltransferase.

17. The composition of claim 16, wherein said fucosyltransferase is a human α(1,2) fucosyltransferase.

18. The composition of claim 1, wherein the genus of said yeast cell is Kluyveromyces.

19. A composition comprising a yeast cell that displays a non-native glycan on its cell surface.

20. The composition of claim 19, wherein said non-native glycan comprises a αfucosylated glycan.

21. The composition of claim 19, wherein said non-native glycan comprises a sialyl glycan.

22. The composition of claim 19, wherein the genus of said yeast cell is Kluyveromyces or Saccharomyces.

23. The composition of claim 19, wherein said yeast cell is a Kluyveromyces lactis yeast cell.

24. A method for producing yeast containing GDP-fucose comprising culturing the yeast of claim 1 under conditions in which said encoded GDP-mannose-4,6-dehydratase and said encoded GDP-L-fucose synthetase are expressed.

25. A method for producing yeast containing GDP-fucose comprising culturing the yeast of claim 1 under conditions in which said encoded GDP-mannose-4,6-dehydratase and said encoded GDP-L-fucose synthetase are expressed simultaneously with at least one overexpressed enzyme selected from the group consisting of phosphomannose isomerase, phosphomannose mutase, and GDP-mannose pyrophosphorylase.

26. A method for producing yeast that are competent for the Golgi import of both GDP-fucose and UDP-galactose comprising culturing the yeast of claim 4 under conditions in which said encoded nucleotide sugar transporter is expressed.

27. A method for producing yeast lacking terminal α(1,2) GlcNAc mannans comprising introducing the Kluyveromyces lactis yeast cell mnn2-1 or mnn2-2 mutation into the yeast cell of claim 6.

28. A method of producing yeast with α(1,2) galactose residues on surface polymannose, comprising culturing the yeast of claim 12 under conditions in which the encoded α(1,2) glycosyltransferase is expressed.

29. A method of producing yeast with β(1,3) galactose linked to surface α(1,2) galactose residues comprising culturing the yeast of claim 14 under conditions in which the β(1,3) glycosyltransferase is expressed.

30. A method of producing yeast with α(1,2) fucosylated glycans on its cell surface comprising culturing the yeast of claim 16 under conditions in which the fucosyltransferase is expressed.

31. A method of treating, preventing, or reducing the risk of infection in a subject comprising administering to said subject a composition comprising an exogenous glycan, wherein said glycan binds to a pathogen and wherein said subject is infected with or at risk of infection with said pathogen.

32. The method of claim 31, wherein said infection is caused by a Norwalk-like virus or Campylobacter jejuni.

33. The method of claim 31, wherein said subject is a mammal.

34. The method of claim 31, wherein said subject is a human.

35. The method of claim 31, wherein said subject is a pig, horse, chicken, cow, sheep, goat, dog, or cat.


SEQUENCE LISTING (24 sequences)

SEQ ID NO: 1 - protein, 381 aa, Helicobacter pylori
MKEKIALITGVTGQDGSYLAEYLLNLGYEVHGLKRRSSSINTSRIDHLYEDLHSDHKRRFFLHYGDMTDSSNLIHLIATTKPTEIYNLAAQSHVKVSFETPEYTANADGIGTLRILEAMRILGLEKKTRFYQASTSELYGEVLETPQNENTPFNPRSPYAVAKMYAFYITKNYREAYNLFAVNGILFNHESRVRGETFVTRKITRAASTIAYNLTDCLYLGNLDAKRDWGHAKDYVKMMHLMLQAPTPQDYVIATGKTTSVRDFVKMSFEFIGINLEFQNTGIKEIGLIKSVDEKRANALQLNLSHLKTGQIVVRIDEHYFRPTEVDLLLGDPTKAEKELGWVREYDLKELVKDMLEYDLKECQKNLYLQDGGYILRNFYE

SEQ ID NO: 2 - protein, 372 aa, Escherichia coli
MTKVALITGVTGQDGSYLAEFLLEKGYEVHGIKRRASSFNTERVDHIYQDPHSAKPNFHLHYGDLTDTSNITRILQEVKPDEVYNLGAMSHVAVSFESPEYTADVDAIGTLRLLEAIRILGLEKKTRFYQASTSELYGLVQEIPQKETTPFYPRSPYAVAKLYAYWITVNYRESYGIYACNGILFNHESPRRGETFVTRKITRAIANIAQGLESCLYLGNMDSLRDWGHAKDYVRMQWMMLQQEQPEDFVIATGVQYSVREFVEMTAEQLGIKLSFEGKGVDEKGIVVSVTGDKAPGVKTGDVIVAVDPRYFRPAEVETLLGDPAKAHEKLGWKPEITLREMISEMVANDLQTAKKHVLLKSHGFNANLAQE

SEQ ID NO: 3 - protein, 372 aa, Homo sapiens
MAHAPARCPSARGSGDGEMGKPRNVALITGITGQDGSYLAEFLLEKGYEVHGIVRRSSSFNTGRIEHLYKNPQAHIEGNMKLHYGDLTDSTCLVKIINEVKPTEIYNLGAQSHVKISFDLAEYTADVDGVGTLRLLDAVKTCGLINSVKFYQASTSELYGKVQEIPQKETTPFYPRSPYGAAKLYAYWIVVNFREAYNLFAVNGILFNHESPRRGANFVTRKISRSVAKIYLGQLECFSLGNLDAKRDWGHAKDYVEAMWLMLQNDEPEDFVIATGEVHSVREFVEKSFLHIGKTIVWEGKNENEVGRCKETGKVHVTVDLKYYRPTEVDFLQGDCTKAKQKLNWKPRVAFDELVREMVHADVELMRTNPNA

SEQ ID NO: 4 - protein, 310 aa, Helicobacter pylori
MNEIILITGAYGMVGQNTALYFKKNKPDVTLLTPKKSELCLLDKDNVQAYLKEYKPTGIIHCAGRVGGIVANMNDLSTYMVENLLMGLYLFSSALDLGVKKAINLASSCAYPKYAPNPLKESDLLNGSLEPTNEGYALAKLSVMKYCEYVSTEKGGFYKTLVPCNLYGEFDKFEEKIAHMIPGLIARMHTAKLKNEKNFAMWGDGTARREYLNAKDLARFIALAYENIASMPSVMNVGSGVDYSIEEYYEMVAQVLDYKGVFVKDLSKPVGMQQKLMDISKQKALKWELEIPLEQGIKEAYEYYLKLLEV

SEQ ID NO: 5 - protein, 321 aa, Escherichia coli
MSKQRVFIAGHRGMVGSAIRRQLEQRGDVELVLRTRDELNLLDSRAVHDFFASERIDQVYLAAAKVGGIVANNTYPADFIYQNMMIESNIIHAAHQNDVNKLLFLGSSCIYPKLAKQPMAESELLQGTLEPTNEPYAIAKIAGIKLCESYNRQYGRDYRSVMPTNLYGPHDNFHPSNSHVIPALLRRFHEATAQNAPDVVVWGSGTPMREFLHVDDMAAASIHVMELAHEVWLENTQPMLSHINVGTGVDCTIRDVAQTIAKVVGYKGRVVFDASKPDGTPRKLLDVTRLHQLGWYHEISLEAGLASTYQWFLENQDRFRG

SEQ ID NO: 6 - protein, 321 aa, Homo sapiens
MGEPQGSMRILVTGGSGLVGKAIQKVVADGAGLPGEDWVFVSSKDADLTDTAQTRALFEKVQPTHVIHLAAMVGGLFRNIKYNLDFWRKNVHMNDNVLHSAFEVGARKVVSCLSTCIFPDKTTYPIDETMIHNGPPHNSNFGYSYAKRMIDVQNRAYFQQYGCTFTAVIPTNVFGPHDNFNIEDGHVLPGLIHKVHLAKSSGSALTVWGTGNPRRQFIYSLDLAQLFIWVLREYNEVEPIILSVGEEDEVSIKEAAEAVVEAMDFHGEVTFDTTKSDGQFKKTASNSKLRTYLPDFRFTPFKQAVKETCAWFTDNYEQARK

SEQ ID NO: 7 - protein, 361 aa, Kluyveromyces lactis
MKGLILVGGYGTRLRPLTLTVPKPLVEFGNRPMILHQIEALAAAGVTDIVLAVNYRPEVMVETLKKYEDEFGVSITFSVETEPLGTAGPLKLAESVLKKDNSPFFVLNSDVICDYPFKELADFHQAHGGKGTIVATKVDEPSKYGVIVHDIATPNLIDRFVEKPVEFVGNRINAGLYILNPEVIDLIDLKPTSIEKETFPILVEQKSLYSFDLEGYWMDVGQPKDFLSGTVLYLNSLSKRDPAKLAKGENIVGNVLVDPTAKISPTAKVGPDVVIGPNVVIGDGVRITRSVALSNSHIKDHALVKSTIIGWNSTVGKWARLEGVTVLGDDVEVKDEIYINGGKVLPHKSISVNVPKEAIIM

SEQ ID NO: 8 - protein, 428 aa, Kluyveromyces lactis
MPQLFRLDAGFQQYDWGKIGSSSAVAQFAAHSDPSVKIDEQKPYAELWMGTHHKVPSYNHDTKESLRDLIEADPVGMLGQGNVDKFGSMKELPFLFKVLSIKKVLSIQAHPDKALAKVLHFNDPANYPDDNHKPEMALAVTDFEGFCGFKPLEEIADELQRIPELRNIVGDEVSETFINNINPEAVKDSADDAKNKKLLQQVFSKVMNASDKVVVENARALIKRAHESPADFNKDTLPQLLIDLNEQFPDDVGLFCGGLLLNHCNLKAGEAIFLRAKDPHAYISGDIIECMAASDNVVRAGFTPKFKDVKNLVEMLTYTYDSVEKQKMSPENFERSSGQGKSVLFNPPIEEFAVLYTTFQDGVGTRHFEGLHGPSIVITTKGNGFIKTGDLKLKAEPGFVFFIAPGTEVDFIADDTDFTTYRAFVEPN

SEQ ID NO: 9 - protein, 254 aa, Kluyveromyces lactis
MSVSEFSHKEQPTTLVLFDVDGTLTPARLTVSDEVRDILKRLREKVVIGFVGGSDLSKQLEQLGSDVLDQFDYAFSENGLTAYRLGKELASQSFIEWIGEEEYNKLAKFILQYLASIDLPKRRGTFLEFRNGMINVSPIGRNASTAERNEFEQFDKEHQVRAKFVEALKKEFAHLSLTFSIGGQISFDVFPTGWDKTYCLRHVEADGFKEIHFFGDKTYKGGNDYEIYVDDRTIGHSVESPADTVRILKELFDL

SEQ ID NO: 10 - protein, 364 aa, Homo sapiens
MNRAPLKRSRILHMALTGASDPSAEAEANGEKPFLLRALQIALVVSLYWVTSISMVFLNKYLLDSPSLRLDTPIFVTFYQCLVTTLLCKGLSALAACCPGAVDFPSLRLDLRVARSVLPLSVVFIGMITFNNLCLKYVGVAFYNVGRSLTTVFNVLLSYLLLKQTTSFYALLTCGIIIGGFWLGVDQEGAEGTLSWLGTVFGVLASLCVSLNAIYTTKVLPAVDGSIWRLTFYNNVNACILFLPLLLLLGELQALRDFAQLGSAHFWGMMTLGGLFGFAIGYVTGLQIKFTSPLTHNVSGTAKACAQTVLAVLYYEETKSFLWWTSNMMVLGGSSAYTWVRGWEMKKTPEEPSPKDSEKSAMGV

SEQ ID NO: 11 - protein, 393 aa, Mus musculus
MAAVGVGGSTAAAGAGAVSSGALEPGSTTAAHRRLKYISLAVLVVQNASLILSIRYARTLPGDRFFATTAVVMAEVLKGLTCLLLLFAQKRGNVKHLVLFLHEAVLVQYVDTLKLAVPSLIYTLQNNLQYVAISNLPAATFQVTYQLKILTTALFSVLMLNRSLSRLQWASLLLLFTGVAIVQAQQAGGSGPRPLDQNPGAGLAAVVASCLSSGFAGVYFEKILKGSSGSVWLRNLQLGLFGTALGLVGLWWAEGTAVASQGFFFGYTPAVWGVVLNQAFGGLLVAVVVKYADNILKGFATSLSIVLSTVASIRLFGFHLDPLFALGAGLVIGAVYLYSLPRGAVKAIASASASGPCIHQQPPGQPPPPQLSSRGDLTTEPFLPKLLTKVKGS

SEQ ID NO: 12 - protein, 393 aa, Homo sapiens
MAAVGAGGSTAAPGPGAVSAGALEPGTASAAHRRLKYISLAVLVVQNASLILSIRYARTLPGDRFFATTAVVMAEVLKGLTCLLLLFAQKRGNVKHLVLFLHEAVLVQYVDTLKLAVPSLIYTLQNNLQYVAISNLPAATFQVTYQLKILTTALFSVLMLNRSLSRLQWASLLLLFTGVAIVQAQQAGGGGPRPLDQNPGAGLAAVVASCLSSGFAGVYFEKILKGSSGSVWLRNLQLGLFGTALGLVGLWWAEGTAVATRGFFFGYTPAVWGVVLNQAFGGLLVAVVVKYADNILKGFATSLSIVLSTVASIRLFGFHVDPLFALGAGLVIGAVYLYSLPRGAAKAIASASASASGPCVHQQPPGQPPPPQLSSHRGDLITEPFLPKSVLVK

SEQ ID NO: 13 - protein, 353 aa, Schizosaccharomyces pombe
MAVKGDDVKWKGIPMKYIALVLLTVQNSALILTLNYSRIMPGYDDKRYFTSTAVLLNELIKLVVCFSVGYHQFRKNVGKEAKLRAFLPQIFGGDSWKLAIPAFLYTCQNNLQYVAAGNLTAASFQVTYQLKILTTAIFSILLLHRRLGPMKWFSLFLLTGGIAIVQLQNLNSDDQMSAGPMNPVTGFSAVLVACLISGLAGVYFEKVLKDTNPSLWVRNVQLSFFSLFPCLFTILMKDYHNIAENGFFFGYNSIVWLAILLQAGGGIIVALCVAFADNIMKNFSTSISIIISSLASVYLMDFKISLTFLIGVMLVIAATFLYTKPESKPSPSRGTYIPMTTQDAAAKDVDHKH

SEQ ID NO: 14 - protein, 339 aa, Saccharomyces cerevisiae
MAGSTSSLVICAIGIYATFLTWALVQEPLATRTWPNSMGKFQFPNVISLIQASVAMMMGYLYLNWKKVEYPPRKMIKDHWKQLMLISFTQSSSGPLATTSLKHVDYLTYMLAKSCKMIPVLLVHLLLYRTPIASQKKVVALLVSLGVTIFTIGGNDGKKLKRSFNESGNDNKLQGFGLLFSSLFLDGLTNATQDKLLKANKAKEKGKQTLITGAHLMFTLNLFVILWNILYFIVIDCKQWDNAVSVLTMDPQVWGYLMLYSFCGAMGQCFIFYTLEQFGSLVLIMITVTRKMVSMILSIIVFGKSVRFQQWVGMFIVFGGITWEALNKKKANIPKAKSA

SEQ ID NO: 15 - protein, 460 aa, Kluyveromyces lactis [sequence omitted]

SEQ ID NO: 16 - protein, 328 aa, Kluyveromyces lactis
MSFVLILSLVFGGCCSNVISFEHMVQGSNINLGNIVTFTQFVSVTLIQLPNALDFSHFPFRLRPRHIPLKIHMLAVFLFFTSSVANNSVFKFDISVPIHIIIRCSGTTLTMIIGWAVCNKRYSKLQVQSAIIMTLGAIVASLYRDKEFSMDSLKLNTDSVGMTQKSMFGIFVVLVATALMSLLSLLNEWTYNKCGKHWKETLFYSHFLALPLFMLGYTRLRDEFRDLLISSDSMDIPIVKLPIATKLFMLIANNVTQFICIKGVNMLASNTDALTLSVVLLVRKFVSLLLSVYIYKNVLSVTAYLGTITVFLGAGLYSYGSVKTALPR

SEQ ID NO: 17 - protein, 375 aa, Schizosaccharomyces pombe
MRFAPYLISAVVITTIILGGAWWTSAMDTKLQTKMKEIIDQHTSTWTPVVSSVTSTQTDTLRVTISEVVSVTATLTETFTATPTVTSVVHALATTDPHPDNSKIVILMGSNFQNDANSPLHPFAQSIIKNRREYAERHGYKFEFLDADAYASRVTGHLMPWVKVPMLQDTMKKYPDAEWIWWLDHDALVMNKDLNVVDHVLKHDRLNTILTREAEYKSGAGIPADGFRTPKDQDAKDVHFIISQDFNGINAGSLFIRNSEVGRWIVDLWFEPLYLDHIQGYAEQQAFSHMVFYHPQVYKHVGVVPLKAINAYDFDDNIWGYDDGDLCIHFAGCNYFKNCPEKFLKYAQILSSKQGSDWMSAQEKDHIQNLLKPSS

SEQ ID NO: 18 - protein, 378 aa, Schizosaccharomyces pombe
MFSNSKKKIFLYVLIAGVATFSFAFLVLNRLQAEEHSLAYVENLFLDPFIKQNESLAHANDRPFKLYLGIFSQAKNVDRRNFLRTDYNEYIKEFAVNDTVDVRFILGLPENEQELATIREEQRTYGDLAVLPIPENVDAGKSIVYFQTFLEGYQPFPLFSELADNLIMPSTQFHGSFIYNQSIKTYELPGMKEFQDLGEPKHDYDFIVKADDDSFLNLPRLFEMLKEHVGKSRFYFGRDCTRRELPTAVRDFPYMCGFFYIVSPDMAYEVAKRRNIIIPFEDAQTGYSIYLSGNVKNAEFSKCTLYDLILPNEGFNYRQSYLRIDAIAVHKLKSIPLLSTVSNWFKKMYEHRANCSALIETERLSCLQATIPLPSLDV

SEQ ID NO: 19 - protein, 365 aa, Homo sapiens
MWLRSHRQLCLAFLLVCVLSVIFFLHIHQDSFPHGLGLSILCPDRRLVTPPVAIFCLPGTAMGPNASSSCPQHPASLSGTWTVYPNGRFGNQMGQYATLLALAQLNGRRAFILPAMHAALAPVFRITLPVLAPEVDSRTPWRELQLHDWMSEEYADLRDPFLKLSGFPCSWTFFHHLREQIRREFTLHDHLREEAQSVLGQLRLGRTGDRPRTFVGVHVRRGDYLQVMPQRWKGVVGDSAYLRQAMDWFRARHEAPVFVVTSNGMEWCKENIDTSQGDVTFAGDGQEATPWKDFALLTQCNHTIMTIGTFGFWAAYLAGGDTVYLANFTLPDSEFLKIFKPEAAFLPEWVGINADLSPLWTLAKP

SEQ ID NO: 20 - protein, 343 aa, Homo sapiens
MLVVQMPFSFPMAHFILFVFTVSTIFHVQQRLAKIQAMWELPVQIPVLASTSKALGPSQLRGMWTINAIGRLGNQMGEYATLYALAKMNGRPAFIPAQMHSTLAPIFRITLPVLHSATASRIPWQNYHLNDWMEEEYRHIPGEYVRFTGYPCSWTFYHHLRQEILQEFTLHDHVREEAQKFLRGLQVNGSRPGTFVGVHVRRGDYVHVMPKVWKGVVADRRYLQQALDWFRARYSSLIFVVTSNGMAWCRENIDTSHGDVVFAGDGIEGSPAKDFALLTQCNHTIMTIGTFGIWAAYLTGGDTIYLANYTLPDSPFLKIFKPEAAFLPEWTGIAADLSPLLKH

SEQ ID NO: 21 - protein, 300 aa, Helicobacter pylori
MAFKVVQICGGLGNQMFQYAFAKSLQKHSNTPVLLDITSFDWSDRKMQLELFPINLPYASAKEIAIAKMQHLPKLVRDALKCMGFDRVSQEIVFEYEPELLKPSRLTYFYGYFQDPRYFDAISPLIKQTFTLPPPPENNKNNNKKEEEYHRKLSLILAAKNSVFVHIRRGDYVGIGCQLGIDYQKKALEYMAKRVPNMELFVFCEDLEFTQNLDLGYPFMDMTTRNKEEEAYWDMLLMQSCQHGIIANSTYSWWAAYLIENPEKIIIGPKHWLFGHENILCKEWVKIESHFEVKSQKYNA

SEQ ID NO: 22 - DNA, 8236 bp, Kluyveromyces lactis
tttaaacgctttttctttccaattttttttttttcgtcattatagaaatcattacgaccgagattcccgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagctagggagcccttgcatgacaattctgctaacatcaaaaggcctctaggttcctttgttacttcttccgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacagtcgactgtgctccttccttcgttcttccttctgctcggagattaccgaatcaaaaaaatttcaaagaaaccggaatcaaaaaaaagaacaaaaaaaaaaaagatgaattgaaaagcgcggccgtgcacaaacgaacgtctcacttaatcttctgtactctgaagaggagtgggaaataccaagaaaaacatcaaactcgaatgattttcccaaacccctaccacaagatattcatcagctgcgagataggctgatcaggagcaagctcgtacgagaagaaacaaaatgacaaaaaaaatcctatactatataggttacaaataaaaaagtatcaaaaatgaagcctgcatctctcaggcaaatggcattctgacatcctcttgaggatctctcgagtttatgactccagcgcgatcgccacgtcgtagccgtgagatttcagcagagagtgttttttcgccgcttcgaggtcattagccaccatttcagacaccatctctctgagggtgatttccggtttccagcccagtttttcgtgcgctttggtcgggtcgccgagcagcgtttcaacttcagccggacggaagtaacgcgggtcaacagcgataatcacatcacccggtttaacgcccggcgcgtcatgcccggtgacggaaaccacaatgcccttctcttcaacgcccgtgccttcaaagcgcagtttgatgcccagctgtgctgccgccatttccacgaactgacgcacggagtactgaacgccggtcgcgataacgaaatcttccggctgttcctgctgcagcatcatccactgcatttttacgtagtctttggcgtggccccagtcacgcagggaatccatattgccgaggtacaggcacgactccagcccctgggcgatgttggcgattgcgcgggtgattttgcgggtaacgaaggtttcgccgcggcgcggggattcatggttgaagagaattccgttacaggcgtacatgccgtaggattcacggtagttaacggtgatccagtaggcgtacagtttggcgaccgcatacggagatcgcgggtagaacggcgtggtctctttctgcggaatttcctgcaccagaccatacagttcagaggtggaagcctgatagaaacgagttttcttttccagaccgaggaagcggatcgcctccagcaggcgcagcgtacccatcgcgtcgacgtcagcggtatattctggtgactcaaaagagaccgcaacgtggctcattgcgcccaggttgtacacttcatccggctgtacttcacgcaaaatgcgcgtcaggttagaggtatcactcaggtcgccataatgcagatggaatttcgggttgcaggtgtgcggatcctgataaatgtgatccacgcgctcggtgttgaatgacgatgcgcgacgcttaataccatgcacctcgtaacctttttccagcagaaactctgccaggtaagaaccgtcttgtccggttacaccggtgatgagagcgacttttgacatttttttcaagcttactagtggatccagatcagcagtagctctctgtgcctcttttcctctatcctgttttatgcaaaatgtgctatcgtaatggtaaaattcaatggcttaaaggttgaaattttatttcaaaagattcacagcttttcctctttacaatgtaaaaattttttttttcttctttcgtgtatattgagaagattgacatgatggtcaagtttatgacgagatgagatgcgatgagggggaaaaagagaggatctgcggccgtgcacaaacgaacgtctcacttaatcttctgtactctgaagaggagtgggaaataccaagaaaaacatcaaactcgaatgattttcccaaacccctaccacaagatattcatcagctgcgagataggctgatcaggagcaagctcgtacgagaagaaacaaaatgacaaaaaaaatcctatactatataggttacaaataaaaaagtatcaaaaatgaagcctgcatctctcaggcaaatggcattctgacatcctcttgaggatctctcgagtttacccccgaaagcggtcttgattctcaaggaaccactggtaagtgctggcaagccccgcttccagtgagatttcgtgataccagccaagctgatgcaggcgcgtcacatccagcagtttgcgcggcgtgccatccggtttgctggcatcaaaaaccacccggcctttgtaacccaccactttggcgatggtttgcgccagctcgcggatagtgcagtcaacgcccgtgccgacgttaatgtgcgacaacatcggctgggtgttctccagccagacttcatgcgccagctccatgacatgaatgctcgccgccgccatatcatcgacgtgcagaaattcgcgcatcggtgtaccgctgccccataccaccacgtccggcgcattctgtgccgtcgcctcgtggaagcgacgcagcaatgctgggatcacatgcgaattactcgggtggaagttgtcgtgtggcccgtacaggttggtcggcatgactgagcggtaatcgcgtccgtactggcggttgtatgattcgcacagtttgatcccggcgattttggcaatagcataaggctcgttagtcggctccagcgtgccctgcaacaactcgctttctgccatcggctgttttgccagtttcgggtagatgcaggacgatccgagaaacagcagtttgttcacgtcgttctgatgcgcggcgtgaatgatgttgctctcaatcatcatgttctggtagatgaaatccgccggataggtgttgttggcaacaatgccgcccactttcgccgccgccagatagacctggtcaatacgttcgctggcaaagaaatcatgcacggcgcggctgtccagcaggttcagctcgtcgcgggtgcgtaataccagttccacatcaccgcgctgttcgagctgccgcctgatggcggaaccgaccatcccgcgatgaccagcaataaaaactcgttgtttactcatttttttcaagcttactagtggatccagatcggacgggaaacggtgctttctggtagatatggccgcaaccgaaatcttaagcagttacagtggtagtagttgtgtctggcttacggttgtagtatgttgttaggccaatttatttaggaagtgggctagctaactttattgtttgcttagatatattttttcaattatttttcattttttcatttttttcatttttttatttttttatttttttatttttttataattggcttcttgtgggatgtatagttttttttatttttcataccggatctaatacttgcaaacgaccgtacttcaatcgtagtggttctatatgtgctatatttgaaatcgaagatatttctgccgttgcatcatctagccttttctcgataagatctgcggccgcatctacacgaagagggaaaattgactcgtgccaggaaacttgaccgtaccactcttttccataccgttagagatgttttacttcatgatgggtcttacacccgtcacaaaggggtccgttgtttggacaacggtagcgttagagtgagcatacggcttaagcaccgtcaattaaccgctacctggatagaattggaacccattattgagaatggcgaggtgaaagacgttatgttcaaattgtccaccagagtacaatatacagaggaaaacgagaacgaaacgcgcccagaatcttcttcagaatgagtgcatgtcagacgcaggcgtcgtatgcttttcattagtgatgcatgccgttgcagcgtgcgaatcggcacggtaatgattcgaaatctctaattataattaccttctgatatatagatgaaagactatttaatgagaatattggacagtaccgttggacttcgctgagaggtacatgggcattttggttattggatgtagaatggttgaataaacgtgattgtaaaatagagtttgtaactacgaataattagtttttgagaagtttggtgaatttaatatttgtatgaggaaagtaaattttaatacctaaataaacaaaaatatatggtacaggaacgcgaggcaacgcgccgatacagggtcaatgggtacacgagagggtgacactaggcgtagaaagtcattagtataaaatacagtggtatatagtagatatttagtttgttttccttttctttttctccaaacgatatcagacatttgtctgataatgaagcattatcagacaaatgtctgatatcgtttttcaataataatatacatcatcacaaaacaaacaaacatagcatcgcaagccccatcatgccaccaccgtccgctgtgatcggcgcgccgcggctacttttcaattccctatagtgagtcgtattaaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctcccgcgg

SEQ ID NO: 23 - DNA, 386 bp, Kluyveromyces lactis
gatcttatcgagaaaaggctagatgatgcaacggcagaaatatcttcgatttcaaatatagcacatatagaaccactacgattgaagtacggtcgtttgcaagtattagatccggtatgaaaaataaaaaaaactatacatcccacaagaagccaattataaaaaaataaaaaaataaaaaaataaaaaaatgaaaaaaatgaaaaaatgaaaaataattgaaaaaatatatctaagcaaacaataaagttagctagcccacttcctaaataaattggcctaacaacatactacaaccgtaagccagacacaactactaccactgtaactgcttaagatttcggttgcggccatatctaccagaaagcaccgtttcccgtccgatc

SEQ ID NO: 24 - DNA, 236 bp, Kluyveromyces lactis
gatcctctctttttccccctcatcgcatctcatctcgtcataaacttgaccatcatgtcaatcttctcaatatacacgaaagaagaaaaaaaaaatttttacattgtaaagaggaaaagctgtgaatcttttgaaataaaatttcaacctttaagccattgaattttaccattacgatagcacattttgcataaaacaggatagaggaaaagaggcacagagagctactgctgatc